(19) United States
(12) Patent Application Publication
(10) Pub. No.: US 2026/0120866 A1
(43) Pub. Date: Apr. 30, 2026

(54) INTELLIGENT MEDICAL DIAGNOSIS SYSTEM HAVING MULTI-PERSON INTERFACE

(71) Applicants: Tri Thuong Nguyen, Rancho Santa Fe, CA (US); Viet Duc Tran, San Diego, CA (US); Thang Hoang Ho, San Diego, CA (US); Phong Thanh Duong, San Diego, CA (US)

(72) Inventors: Tri Thuong Nguyen, Rancho Santa Fe, CA (US); Viet Duc Tran, San Diego, CA (US); Thang Hoang Ho, San Diego, CA (US); Phong Thanh Duong, San Diego, CA (US)

(21) Appl. No.: 18/932,502

(22) Filed: Oct. 30, 2024

(51) Int. Cl. G16H 50/20; G16H 10/20; G16H 10/60; G16H 15/00; G16H 80/00

(52) U.S. Cl. CPC G16H 50/20; G16H 10/20; G16H 10/60; G16H 15/00; G16H 80/00

(57) ABSTRACT

Systems and methods for automated healthcare services include the use of medical facilitators. A purpose of the facilitators is to serve as a human link between patients and electronic medical applications which utilize medical AI. In some embodiments, medical facilitators assist patients with history and physical examination following the guide of an automated and at least partially AI-based medical computing system or application. The medical facilitator may be trained to use the electronic application and to have sufficient basic medical knowledge to follow directives generated by the application, but he or she is not required to have the medical knowledge of a physician. Medical facilitators can provide the history and physical tasks at the same time, and the physician(s) need only to review and approve the results.

### FIELD

Embodiments of this disclosure relate generally to medical diagnosis systems, and more specifically to intelligent medical diagnosis systems having multi-person interfaces.

### BACKGROUND

Current healthcare delivery still mainly requires human to human interactions between the medical providers and patients in clinic, urgent care centers, hospitals or other medical facilities. Efficiency is limited by factors such as the adequate availability of the medical providers to serve a very large number of patients in most clinics, the time available for meaningful encounters, energy and burnout, as well as the knowledge of medical providers. These factors as well as others may limit adequate exchange of information required for appropriate, timely and effective diagnoses and treatment. The lack of adequate time and information acquisition may also lead to incorrect or defensive medical care, which leads to the use of unnecessary medical testing, which increases the cost of medical care and delay timely treatment. In most clinics, these issues also lead to long waiting time, which creates mental and physical stress of both the patients and the providers, which further worsens providers fatigue and burnout.

### SUMMARY

In an embodiment of the disclosure, systems and methods for automated healthcare services include the use of medical facilitators who are assisted by machine learning applications that help to diagnose patients. A purpose of the facilitators is to serve as a human link between patients and electronic medical applications which utilize medical artificial intelligence (AI). In some embodiments, medical facilitators assist patients with history and physical examination following the guide of an automated and at least partially AI-based medical computing system or application. The medical facilitator may be trained to use the electronic application and to have sufficient basic medical knowledge to follow directives generated by the application, but he or she is not required to have the medical knowledge of a physician. In some embodiments, multiple low-to mid-level medical facilitators can provide the history and physical tasks at the same time, and the physician(s) need only to review and approve the results. This process can also be performed at least partially remotely such as by phone at any time, and telemedicine can be provided similar to the care in a clinic.

In an embodiment of the disclosure, a method may employ an electronic device having one or more processors and a display. The method may include receiving, from a first person, first user input describing symptoms experienced by a second person, as well as determining a plurality of candidate diagnoses for the described symptoms, the plurality of candidate diagnoses determined according to a neural network trained to receive the described symptoms as inputs and to generate the candidate diagnoses as outputs. The method may further include generating questions to exclude ones of the candidate diagnoses, and displaying the generated questions to the first person, for responses by the second person. The method may further include receiving, from the first person, second user input describing the responses by the second person, followed by selecting, according to the second user input, a final diagnosis from among the candidate diagnoses. Selection of the final diagnosis is followed by transmitting the final diagnosis for confirmation by a third person. Upon confirmation of the final diagnosis by the third person, instructions are generated for the second person according to the final diagnosis, and the generated instructions are displayed to the first person, where the instructions are generated for execution by the second person.

### BRIEF DESCRIPTION OF THE DRAWINGS

FIG. 1 conceptually illustrates a medical diagnosis system and its operation, in accordance with various examples of the disclosure;

FIG. 2 is a block diagram illustrating the medical diagnosis system of FIG. 1, in accordance with various examples of the disclosure;

FIG. 3A illustrates an exemplary data center system for implementing a medical diagnosis system in accordance with various examples of the disclosure;

FIG. 3B illustrates inference and/or training logic in accordance with various examples of the disclosure;

FIG. 3C illustrates inference and/or training logic in accordance with various examples of the disclosure;

FIG. 4 illustrates an exemplary computer system for implementing a medical diagnosis system in accordance with various examples of the disclosure;

FIG. 5 illustrates a further exemplary computer system for implementing a medical diagnosis system in accordance with various examples of the disclosure;

FIG. 6 illustrates data flow in an exemplary computing pipeline, in accordance with various examples of the disclosure;

FIG. 7 illustrates an exemplary system for training and deploying machine learning models in a computing system, in accordance with various examples of the disclosure;

FIG. 8A illustrates an exemplary process for training machine learning models in accordance with various examples of the disclosure;

FIG. 8B illustrates an exemplary client-server architecture for annotation of training data, in accordance with various examples of the disclosure;

FIG. 9 is a flow chart representing a process for determining and providing medical diagnoses using a multi-person interface, in accordance with various examples of the disclosure;

FIGS. 10A-10C are an exemplary illustration of a process for determining and providing medical diagnoses using a multi-person interface, in accordance with various examples of the disclosure;

FIGS. 11A-11C are an exemplary illustration of a process for determining and providing a diagnosis of acid reflux using a multi-person interface, in accordance with various examples of the disclosure;

FIGS. 12A-12C are an exemplary illustration of a process for determining and providing a diagnosis of pulmonary embolism using a multi-person interface, in accordance with various examples of the disclosure; and

FIGS. 13A-13C are an exemplary illustration of a process for determining and providing a diagnosis of aspiration pneumonia using a multi-person interface, in accordance with various examples of the disclosure;

### DETAILED DESCRIPTION

In the following description of examples, reference is made to the accompanying drawings in which are shown by way of illustration specific examples that can be practiced. It is to be understood that other examples can be used and structural changes can be made without departing from the scope of the various examples.

Embodiments of the present disclosure relate to an intelligent medical diagnosis system that employs an interface for multiple persons. More specifically, exemplary systems employ a medical facilitator person to serve as a human link between patients and application programs of embodiments of the disclosure. In some embodiments, medical facilitators assist patients with history and physical examination following the guide of the application programs, which determine and present a diagnosis of the patient to a physician or other medical professional. Once the medical professional confirms the diagnosis, application programs may further present treatment instructions to the medical facilitator, for treatment of the patient. The medical facilitator may be trained to use the electronic application and to have sufficient basic medical knowledge to follow directives generated by the application, but he or she is not required to have the medical knowledge of a physician.

FIG. 1 conceptually illustrates a medical diagnosis system and its operation, in accordance with various examples of the disclosure. A computational diagnostics system 104 may include an interface for interaction with a medical facilitator (MF) 102 and medical doctor (MD) 108 or other medical professional. The computational diagnostics system 104 may be in electronic communication with a stored electronic health records (EHRs) storage 106.

In operation, the MF 102 may act as an intermediary between the computational diagnostics system 104 and a patient 100, to facilitate entry of patient 100 information into diagnostics system 104 in a manner and form better suited for use by system 104, and explanation of diagnosis results from system 104 to patient 100 in a manner and form more easily understood by patient 100. MFs 102 may enter patient 100 information from patient 100 into diagnostics system 104, describing symptoms and other medical information used by system 104 to generate diagnoses. Diagnostics system 104 may then diagnose patient 100, in some embodiments employing one or more neural networks or other AI-based methods or processes. For example, diagnostics system 104 may retrieve patient 100 medical information from EHR storage 106 as well as from queries to patient 100 through MF 102, and may input corresponding formatted information to one or more neural networks trained to output medical diagnoses from input patient symptoms or other information. Symptoms described by patients 100 and entered into diagnostics system 104 may be any physical or mental symptoms that may be experienced or described by a patient. Physical symptoms may include any outward, observable signs or sensations related to the body's physiological state, including without limitation pain (e.g., headaches or chest pain), fatigue, shortness of breath, fever, or skin rashes. Mental symptoms may include any emotional, cognitive, or behavioral experiences reflecting changes in a person's mental state. Examples may include anxiety, depression, confusion, memory problems, hallucinations, or paranoia.

Neural networks of system 104 may then return output diagnoses. In some embodiments, these diagnoses may be transmitted to an MD 108 for review and confirmation, providing a layer of human expertise for greater reliability and safety of results. Once confirmed, system 104 may display these diagnoses along with stored treatment instructions to MF 102, who may then relay them to patient 100 in a manner more easily understood by patient 100 than instructions from an automated system such as system 104.

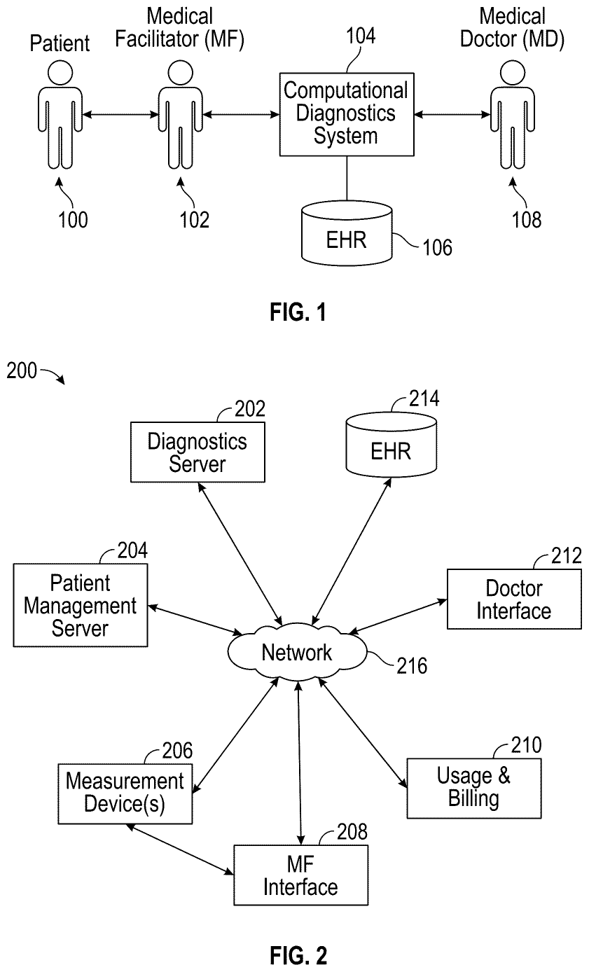

FIG. 2 is a block diagram illustrating the medical diagnosis system of FIG. 1, in accordance with various examples of the disclosure. Here, medical diagnosis system 200 includes a diagnostics server 202, a patient management server 204, one or more measurement devices 206, MF interface 208, usage and billing server 210, a doctor interface 212, and EHR storage 214. Each of these components is in electronic communication with each other via a communications network 216 which may be any telecommunications network such as, for example, the public Internet.

The diagnostics server 202 is described herein as a server computer, but may be any computing device capable of receiving patient 100 data, executing and/or training one or more neural networks to determine patient 100 diagnoses, and communicating these diagnoses to another computing device. In some embodiments, diagnostics server 202 is a server computer residing within a data center, but is not limited to this configuration and may be a standalone server, or any other suitable computing device such as a laptop or desktop computer, portable computing device, or the like.

Similarly, patient management server 204 may be any computing device capable of executing one or more interface applications for exchanging information between MF 102 and diagnostics server 202. For example, patient management server 204 may execute interface programs for displaying queries to MF 102 requesting patient 100 information, receiving resulting information from the patient 100 and entered by MF 102, and transmitting the received information to diagnostics server 202. These interface programs may also receive diagnoses and treatment instructions from diagnostics server 202 and display them for MF 102 to treat or relay to patient 100. In some embodiments, patient management server 204 is a server computer residing within a data center, but is not limited to this configuration and may be a standalone server, or any other suitable computing device such as a laptop or desktop computer, portable computing device, or the like.

Measurement devices 206 may be any devices for taking measurements of physical properties of patient 100, and may be connected devices capable of transmitting measurements to diagnostics server 202 or another device via network 216. Alternatively, measurement devices 206 may be any standalone devices which must be operated by MF 102 or patient 100 and which rely on MF 102 to input resulting measurements to, e.g., an MF interface 208 for transmission to another device (e.g., diagnostics server 202 or patient management server 204) via network 216. Measurement devices 206 may include imaging devices such as x-ray machines, MRI machines, or the like, with images generated by these machines providing one or more inputs to neural networks of embodiments of the disclosure. MF interface 208 may be any computing device capable of receiving measurement data entered by MF 102 and transmitting it to another device via network 216. In some embodiments, MF interface 208 is integrated into the functionality of one or more application programs of patient management server 204.

Usage and billing server 210 may be any computing device capable of executing one or more usage and billing applications, and electronically communicating with any one or more of diagnostics server 202, patient management server 204, and EHR storage 214. In some embodiments, usage and billing applications executed by server 210 track and process billing information related to patient 100 visits for diagnosis by MF 102 and diagnostics server 202, and related to treatments determined by diagnostics server 202. Usage and billing server 210 may be any computing device capable of executing one or more usage and billing applications, and exchanging corresponding information with one or more of diagnostics server 202, patient management server 204, and EHR storage 214 over network 216. In some embodiments, usage and billing applications executed by usage and billing server 210 are integrated into the functionality of one or more application programs of patient management server 204. In some embodiments, usage and billing server 210 is a server computer residing within a data center, but is not limited to this configuration and may be a standalone server, or any other suitable computing device such as a laptop or desktop computer, portable computing device, or the like.

Doctor interface 212 may be any computing device capable of executing one or more application programs allowing an MD 108 to view and confirm diagnoses of patient 100 by diagnostics server 202. In various embodiments, doctor interface 212 may be a server computer residing within a data center, but is not limited to this configuration and may be a standalone server located onsite with MD 108, or any other suitable computing device such as a laptop or desktop computer, portable computing device, or the like.

EHR storage 214 may store electronic patient records for any number of patients 100. Records and/or any information therein may be transmitted to any device, such as diagnostics server 202 or patient management server 204, as desired for determination of diagnoses. For example, treatment or health history information may be requested by patient management server 204 for transmission to diagnostics server 202, to serve as inputs to neural network models to assist in accurate determination of output diagnoses. EHR storage 214 may be any electronic storage device capable of storing, revising, and transmitting electronic patient records and information for retrieval and use by any device, such as diagnostics server 202 and/or patient management server 204. In some embodiments, EHR storage 214 may be a storage device of a data center, such as a disk array. Alternatively, EHR storage 214 may be any other device capable of storing information in digital form, such as a memory or storage of a laptop or desktop computer, portable computing device, or the like.

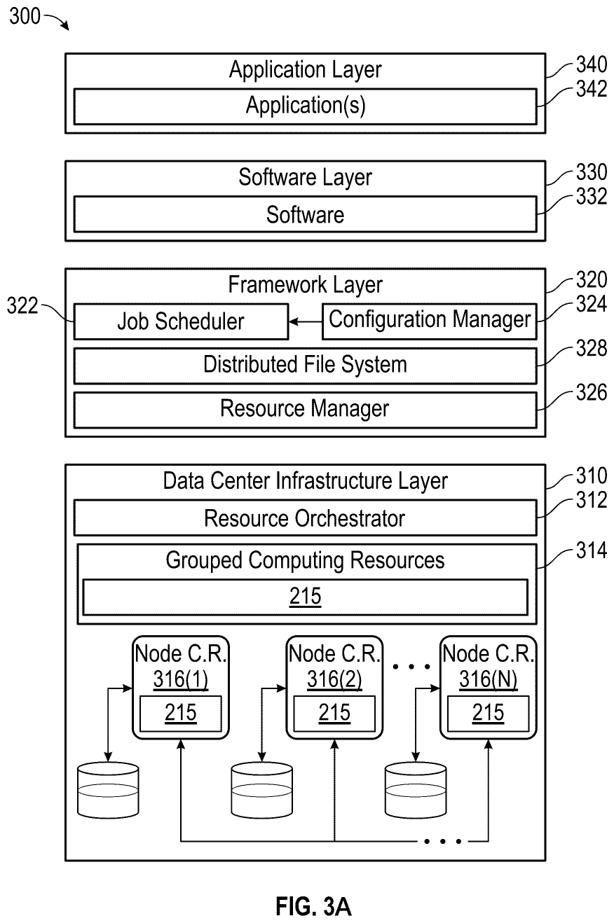

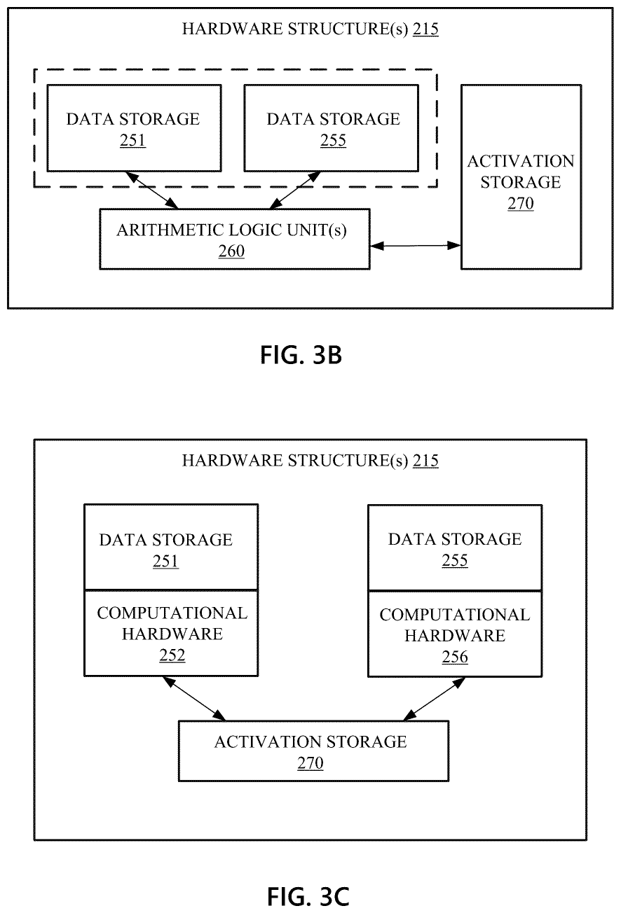

FIG. 3A illustrates an exemplary data center system for implementing a medical diagnosis system in accordance with various examples of the disclosure. In embodiments of the disclosure, data center 300 includes a data center infrastructure layer 310, a framework layer 320, a software layer 330, and an application layer 340. Data center infrastructure layer 310 may include a resource orchestrator 312, grouped computing resources 314, and node computing resources (“node C.R.s”) 316(1)-316(N), where N represents any whole, positive integer. Node C.R.s 316(1)-316(N) may include, but are not limited to, any number of central processing units (“CPUs”) or other processors (including accelerators, field programmable gate arrays (FPGAs), graphics processors, etc.), memory devices (e.g., dynamic read-only memory), storage devices (e.g., solid state or disk drives), network input/output (“NW VO”) devices, network switches, virtual machines (“VMs”), power modules, and cooling modules, etc. One or more node C.R.s from among node C.R.s 316(1)-316(N) may be a server having one or more of above-mentioned computing resources.

Grouped computing resources 314 may include separate groupings of node C.R.s housed within one or more racks (not shown), or many racks housed in data centers at various geographical locations (also not shown). Separate groupings of node C.R.s within grouped computing resources 314 may include grouped compute, network, memory or storage resources that may be configured or allocated to support one or more workloads. Multiple node C.R.s including CPUs or processors may grouped within one or more racks to provide compute resources to support one or more workloads. Racks may also include any number of power modules, cooling modules, and network switches, in any combination. In embodiments employing data center 300, any servers or other computing devices of FIG. 2 may be implemented as node C.R.s 316. For example, diagnostics server 202, patient management server 204, and usage and billing server 210 may each be one or more node C.R.s 316.

Resource orchestrator 312 may configure or otherwise control one or more node C.R.s 316(1)-316(N) and/or grouped computing resources 314. Resource orchestrator 312 may include a software design infrastructure (“SDI”) management entity for data center 300. Resource orchestrator 312 may include hardware, software or some combination thereof.

As shown in FIG. 3A, framework layer 320 includes a job scheduler 322, a configuration manager 324, a resource manager 326 and a distributed file system 328. Framework layer 320 may include a framework to support software 332 of software layer 330 and/or one or more application(s) 342 of application layer 340. Software 332 or application(s) 342 may respectively include web-based service software or applications, such as those provided by Amazon Web Services, Google Cloud and Microsoft Azure. Framework layer 320 may be, but is not limited to, a type of free and open-source software web application framework such as Apache Spark™ (hereinafter “Spark”) that may utilize distributed file system 328 for large-scale data processing (e.g., “big data”). Job scheduler 322 may include a Spark driver to facilitate scheduling of workloads supported by various layers of data center 300. Configuration manager 324 may be capable of configuring different layers such as software layer 330 and framework layer 320 including Spark and distributed file system 328 for supporting large-scale data processing. Resource manager 326 may be capable of managing clustered or grouped computing resources mapped to or allocated for support of distributed file system 328 and job scheduler 322. Clustered or grouped computing resources may include grouped computing resource 314 at data center infrastructure layer 310. In at least one embodiment, resource manager 326 may coordinate with resource orchestrator 312 to manage these mapped or allocated computing resources.

Software 332 included in software layer 330 may include software used by at least portions of node C.R.s 316(1)-316(N), grouped computing resources 314, and/or distributed file system 328 of framework layer 320. One or more types of software may include, but are not limited to, software for executing inferencing operations using trained neural networks as described herein, user interface software, Internet web page search software, e-mail virus scan software, database software, and streaming video content software.

Application(s) 342 included in application layer 340 may include one or more types of applications used by at least portions of node C.R.s 316(1)-316(N), grouped computing resources 314, and/or distributed file system 328 of framework layer 320. One or more types of applications may include, but are not limited to, any number of an MF interface, a doctor interface, a cognitive compute, and a machine learning application, including training or inferencing software, machine learning framework software (e.g., PyTorch, TensorFlow, Caffe, etc.) or other machine learning applications used in conjunction with one or more embodiments.

Any of configuration manager 324, resource manager 326, and resource orchestrator 312 may implement any number and type of self-modifying actions based on any amount and type of data acquired in any technically feasible fashion. Self-modifying actions may relieve a data center operator of data center 300 from making possibly bad configuration decisions and possibly avoiding underutilized and/or poor performing portions of a data center.

Data center 300 may include tools, services, software or other resources to train one or more machine learning models or predict or infer information using one or more machine learning models according to one or more embodiments described herein. For example, a machine learning model may be trained by calculating weight parameters according to a neural network architecture using software and computing resources described above with respect to data center 300. Trained machine learning models corresponding to one or more neural networks may be used to infer or predict information using resources described above with respect to data center 300 by using weight parameters calculated through one or more training techniques described herein.

Data center 300 may use CPUs, application-specific integrated circuits (ASICs), GPUs, FPGAs, or other hardware to perform training and/or inferencing using above-described resources. Moreover, one or more software and/or hardware resources described above may be configured as a service to allow users to train or performing inferencing of information, such as image recognition, speech recognition, or other artificial intelligence services.

Inference and/or training logic 215 are used to perform inferencing and/or training operations associated with one or more embodiments. Details regarding inference and/or training logic 215 are provided below in conjunction with FIGS. 3B-3C. In at least one embodiment, inference and/or training logic 215 may be used in the system of FIG. 3A for inferencing or predicting operations based, at least in part, on weight parameters calculated using neural network training operations, neural network functions and/or architectures, or neural network use cases described herein.

Inference and/or training logic 215 are used to perform inferencing and/or training operations associated with one or more embodiments. This logic can be used with components of these figures to train one or more neural networks based, at least in part, on input data such as patient symptoms, states, or conditions and their corresponding diagnoses.

Details regarding inference and/or training logic 215 are provided below in conjunction with FIGS. 3B and/or 3C. Inference and/or training logic 215 may include, without limitation, code and/or data storage 251 to store forward and/or output weight and/or input/output data, and/or other parameters to configure neurons or layers of a neural network trained and/or used for inferencing in aspects of one or more embodiments. Training logic 215 may include, or be coupled to code and/or data storage 251 to store graph code or other software to control timing and/or order, in which weight and/or other parameter information is to be loaded to configure, logic, including integer and/or floating point units (collectively, arithmetic logic units (ALUs). Code, such as graph code, loads weight or other parameter information into processor ALUs based on architecture of a neural network to which this code corresponds. Code and/or data storage 251 stores weight parameters and/or input/output data of each layer of a neural network trained or used in conjunction with one or more embodiments during forward propagation of input/output data and/or weight parameters during training and/or inferencing using aspects of one or more embodiments. Any portion of code and/or data storage 251 may be included with other on-chip or off-chip data storage, including a processor's LI, L2, or L3 cache or system memory.

Any portion of code and/or data storage 251 may be internal or external to one or more processors or other hardware logic devices or circuits. Code and/or data storage 251 may be cache memory, dynamic randomly addressable memory (“DRAM”), static randomly addressable memory (“SRAM”), non-volatile memory (e.g., Flash memory), or other storage. Choice of whether code and/or data storage 251 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

Inference and/or training logic 215 may include, without limitation, a code and/or data storage 255 to store backward and/or output weight and/or input/output data corresponding to neurons or layers of a neural network trained and/or used for inferencing in aspects of one or more embodiments. Code and/or data storage 255 stores weight parameters and/or input/output data of each layer of a neural network trained or used in conjunction with one or more embodiments during backward propagation of input/output data and/or weight parameters during training and/or inferencing using aspects of one or more embodiments. Training logic 215 may include, or be coupled to code and/or data storage 255 to store graph code or other software to control timing and/or order, in which weight and/or other parameter information is to be loaded to configure, logic, including integer and/or floating point units (collectively, arithmetic logic units (ALUs). Code, such as graph code, loads weight or other parameter information into processor ALUs based on an architecture of a neural network to which this code corresponds. Any portion of code and/or data storage 255 may be included with other on-chip or off-chip data storage, including a processor's LI, L2, or L3 cache or system memory. Any portion of code and/or data storage 255 may be internal or external to on one or more processors or other hardware logic devices or circuits. Code and/or data storage 255 may be cache memory, DRAM, SRAM, non-volatile memory (e.g., Flash memory), or other storage. The choice of whether code and/or data storage 255 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

Inference and/or training logic 215 may include, without limitation, a code and/or data storage 255 to store backward and/or output weight and/or input/output data corresponding to neurons or layers of a neural network trained and/or used for inferencing in aspects of one or more embodiments. Code and/or data storage 255 stores weight parameters and/or input/output data of each layer of a neural network trained or used in conjunction with one or more embodiments during backward propagation of input/output data and/or weight parameters during training and/or inferencing using aspects of one or more embodiments. Training logic 215 may include, or be coupled to code and/or data storage 255 to store graph code or other software to control timing and/or order, in which weight and/or other parameter information is to be loaded to configure, logic, including integer and/or floating point units (collectively, arithmetic logic units (ALUs). Code, such as graph code, loads weight or other parameter information into processor ALUs based on an architecture of a neural network to which this code corresponds. Any portion of code and/or data storage 255 may be included with other on-chip or off-chip data storage, including a processor's LI, L2, or L3 cache or system memory. Any portion of code and/or data storage 255 may be internal or external to on one or more processors or other hardware logic devices or circuits. Code and/or data storage 255 may be cache memory, DRAM, SRAM, non-volatile memory (e.g., Flash memory), or other storage. The choice of whether code and/or data storage 255 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

Code and/or data storage 251 and code and/or data storage 255 may be separate storage structures or may be the same storage structure. Code and/or data storage 251 and code and/or data storage 255 may be partially the same storage structure and partially separate storage structures. Any portion of code and/or data storage 25 land code and/or data storage 255 may be included with other on-chip or off-chip data storage, including a processor's LI, L2, or L3 cache or system memory.

Inference and/or training logic 215 may include, without limitation, one or more arithmetic logic unit(s) (“ALU(s)”) 260, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 270 that are functions of input/output and/or weight parameter data stored in code and/or data storage 251 and/or code and/or data storage 255. Activations stored in activation storage 270 are generated according to linear algebraic and or matrix-based mathematics performed by ALU(s) 260 in response to performing instructions or other code, wherein weight values stored in code and/or data storage 255 and/or code and/or data storage 251 are used as operands along with other values, such as bias values, gradient information, momentum values, or other parameters or hyperparameters, any or all of which may be stored in code and/or data storage 255 or code and/or data storage 251 or another storage on or off-chip.

In some embodiments, ALU(s) 260 may be included within one or more processors or other hardware logic devices or circuits, whereas in another embodiment, ALU(s) 260 may be external to a processor or other hardware logic device or circuit that uses them (e.g., a coprocessor). ALUs 260 may be included within a processor's execution units or otherwise within a bank of ALUs accessible by a processor's execution units either within same processor or distributed between different processors of different types (e.g., central processing units, graphics processing units, fixed function units, etc.). Code and/or data storage 251, code and/or data storage 255, and activation storage 270 may be on the same processor or other hardware logic device or circuit, whereas in another embodiment, they may be in different processors or other hardware logic devices or circuits, or some combination of same and different processors or other hardware logic devices or circuits. Any portion of activation storage 270 may be included with other on-chip or off-chip data storage, including a processor's LI, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

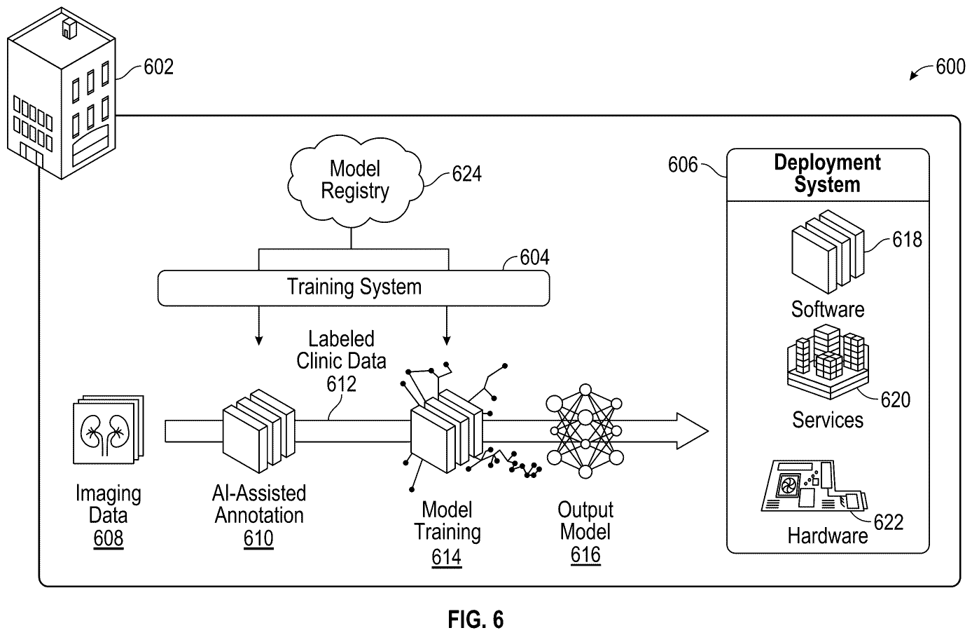

Activation storage 270 may be cache memory, DRAM, SRAM, non-volatile memory (e.g., Flash memory), or other storage. In at least one embodiment, activation storage 270 may be completely or partially within or external to one or more processors or other logical circuits. The choice of whether activation storage 270 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors. Inference and/or training logic 215 illustrated in FIG. 6A may be used in conjunction with an application-specific integrated circuit (“ASIC”), such as Tensorflow® Processing Unit from Google, an inference processing unit (IPU) from Graphcore™, or a Nervana® (e.g., “Lake Crest”) processor from Intel Corp. Inference and/or training logic 215 illustrated in FIG. 6A may be used in conjunction with central processing unit (“CPU”) hardware, graphics processing unit (“GPU”) hardware or other hardware, such as field programmable gate arrays (“FPGAs”).

FIG. 3C illustrates inference and/or training logic 215, according to at least one or more embodiments. Inference and/or training logic 215 may include, without limitation, hardware logic in which computational resources are dedicated or otherwise exclusively used in conjunction with weight values or other information corresponding to one or more layers of neurons within a neural network. Inference and/or training logic 215 illustrated in FIG. 3C may be used in conjunction with an application-specific integrated circuit (ASIC), such as Tensorflow® Processing Unit from Google, an inference processing unit (IPU) from Graphcore™, or a Nervana® (e.g., “Lake Crest”) processor from Intel Corp. Inference and/or training logic 215 illustrated in FIG. 3C may be used in conjunction with central processing unit (CPU) hardware, graphics processing unit (GPU) hardware or other hardware, such as field programmable gate arrays (FPGAs). Inference and/or training logic 215 includes, without limitation, code and/or data storage 251 and code and/or data storage 255, which may be used to store code (e.g., graph code), weight values and/or other information, including bias values, gradient information, momentum values, and/or other parameter or hyperparameter information.

In FIG. 3C, each of code and/or data storage 251 and code and/or data storage 255 may be associated with a dedicated computational resource, such as computational hardware 252 and computational hardware 256, respectively. Each of computational hardware 252 and computational hardware 256 may comprise one or more ALUs that perform mathematical functions, such as linear algebraic functions, only on information stored in code and/or data storage 251 and code and/or data storage 255, respectively, result of which is stored in activation storage 270.

Each of code and/or data storage 251 and 255 and corresponding computational hardware 252 and 256, respectively, correspond to different layers of a neural network, such that resulting activation from one “storage/computational pair 251/252” of code and/or data storage 251 and computational hardware 252 is provided as an input to “storage/computational pair 255/256” of code and/or data storage 255 and computational hardware 256, in order to mirror conceptual organization of a neural network. Each of storage/computational pairs 251/252 and 255/256 may correspond to more than one neural network layer. Additional storage/computation pairs (not shown) subsequent to or in parallel with storage computation pairs 251/252 and 255/256 may be included in inference and/or training logic 215.

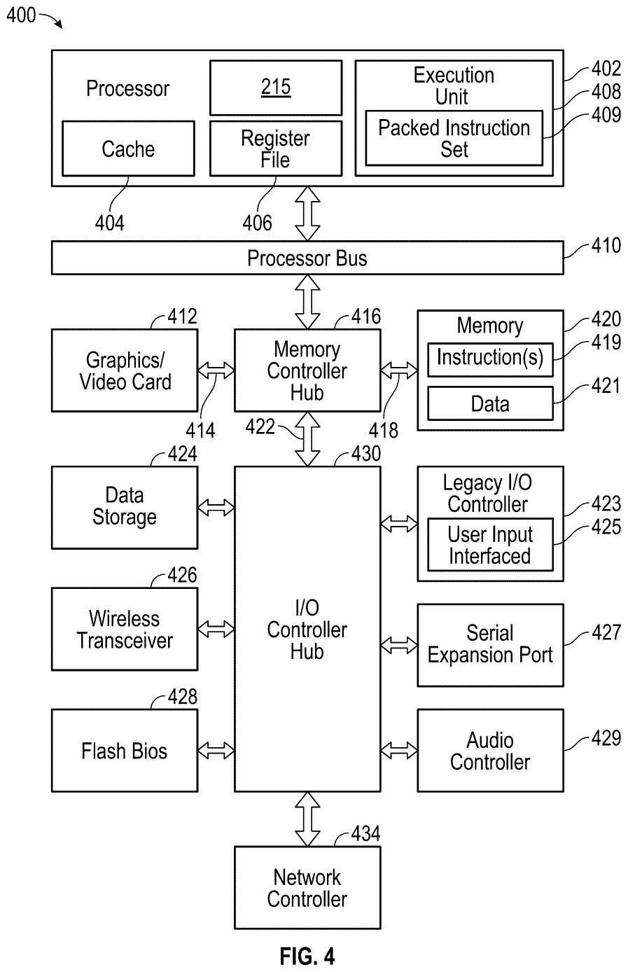

FIG. 4 illustrates an exemplary computer system for implementing a medical diagnosis system in accordance with various examples of the disclosure. Each computing device of FIG. 2 may be implemented as a computer system of FIG. 4. For example, diagnostics server 202, patient management server 204, usage and billing server 210, and doctor interface 212 may each be implemented on a computer system of FIG. 4.

The exemplary computer system of FIG. 4 may be a system with interconnected devices and components, a system-on-a-chip (SOC) or some combination thereof 400 formed with a processor that may include execution units to execute an instruction, according to at least one embodiment. Computer system 400 may include, without limitation, a component, such as a processor 402 to employ execution units including logic to perform algorithms for process data, in accordance with present disclosure, such as in embodiment described herein. Computer system 400 may include processors, such as PENTIUM® Processor family, Xeon™, Itanium®, XScale™ and/or StrongARM™, Intel® Core™, or Intel® Nervana™ microprocessors available from Intel Corporation of Santa Clara, California, although other systems (including PCs having other microprocessors, engineering workstations, set-top boxes and like) may also be used. Computer system 400 may execute a version of a WINDOWS operating system available from Microsoft Corporation of Redmond, Wash., although other operating systems (UNIX and Linux for example), embedded software, and/or graphical user interfaces, may also be used.

Embodiments may be used in other devices such as handheld devices and embedded applications. Some examples of handheld devices include cellular phones, Internet Protocol devices, digital cameras, personal digital assistants (“PDAs”), and handheld PCs. In at least one embodiment, embedded applications may include a microcontroller, a digital signal processor (“DSP”), system on a chip, network computers (“NetPCs”), set-top boxes, network hubs, wide area network (“WAN”) switches, or any other system that may perform one or more instructions in accordance with at least one embodiment.

Computer system 400 may include, without limitation, any number of processors 402 that may include, without limitation, one or more execution units 408 to perform machine learning model training and/or inferencing according to techniques described herein. For example, computer system 400 may be a single processor desktop or server system, but in another embodiments computer system 400 may be a multiprocessor system. Processors 402 may include, without limitation, a complex instruction set computer (“CISC”) microprocessor, a reduced instruction set computing (“RISC”) microprocessor, a very long instruction word (“VLIW”) microprocessor, a processor implementing a combination of instruction sets, or any other processor device, such as a digital signal processor, for example. Processor 402 may be coupled to a processor bus 410 that may transmit data signals between processor 402 and other components in computer system 400.

Processor 402 may include, without limitation, a Level 1 (“LI”) internal cache memory (“cache”) 404. Processor 402 may have a single internal cache or multiple levels of internal cache. Cache memory may reside external to processor 402. Other embodiments may also include a combination of both internal and external caches depending on particular implementation and needs. Register file 406 may store different types of data in various registers including, without limitation, integer registers, floating point registers, status registers, and instruction pointer register.

Execution unit 408, including, without limitation, logic to perform integer and floating point operations, also resides in processor 402. Processor 402 may also include a microcode (“ucode”) read only memory (“ROM”) that stores microcode for certain macro instructions. Execution unit 408 may include logic to handle a packed instruction set 409. By including packed instruction set 409 in an instruction set of a general-purpose processor 402, along with associated circuitry to execute instructions, operations used by many multimedia applications may be performed using packed data in a general-purpose processor 402. Many applications may be accelerated and executed more efficiently by using the full width of a processor's data bus for performing operations on packed data, which may eliminate need to transfer smaller units of data across processor's data bus to perform one or more operations one data element at a time.

Execution unit 408 may also be used in microcontrollers, embedded processors, graphics devices, DSPs, and other types of logic circuits. Computer system 400 may include, without limitation, a memory 420. Memory 420 may be implemented as a Dynamic Random Access Memory (“DRAM”) device, a Static Random Access Memory (“SRAM”) device, flash memory device, or other memory device. Memory 420 may store instruction(s) 419 and/or data 421 represented by data signals that may be executed by processor 402.

Any number of system logic chips may be coupled to processor bus 410 and memory 420. A system logic chip may include, without limitation, a memory controller hub (“MCH”) 416, and processor 402 may communicate with MCH 416 via processor bus 410. MCH 416 may provide a high bandwidth memory path 418 to memory 420 for instruction and data storage and for storage of graphics commands, data and textures. MCH 416 may direct data signals between processor 402, memory 420, and other components in computer system 400 and to bridge data signals between processor bus 410, memory 420, and a system I/O 422. Each system logic chip may provide a graphics port for coupling to a graphics controller. MCH 416 may be coupled to memory 420 through a high bandwidth memory path 418 and graphics/video card 412 may be coupled to MCH 416 through an Accelerated Graphics Port (“AGP”) interconnect 414.

Computer system 400 may use system I/O 422 that is a proprietary hub interface bus to couple MCH 416 to I/O controller hub (“ICH”) 430. ICH 430 may provide direct connections to some I/O devices via a local I/O bus. Local I/O buses may include, without limitation, a high-speed I/O bus for connecting peripherals to memory 420, chipset, and processor 402. Examples may include, without limitation, an audio controller 429, a firmware hub (“flash BIOS”) 428, a wireless transceiver 426, a data storage 424, a legacy VO controller 423 containing user input and keyboard interfaces 425, a serial expansion port 427, such as Universal Serial Bus (“USB”), and a network controller 434. Data storage 424 may comprise a hard disk drive, a floppy disk drive, a CD-ROM device, a flash memory device, or other mass storage device.

FIG. 4 may illustrate a system which includes interconnected hardware devices or “chips.” Alternatively, FIG. 4 may illustrate an exemplary System on a Chip (“SoC”). Devices illustrated in FIG. 4 may be interconnected with proprietary interconnects, standardized interconnects (e.g., PCIe) or some combination thereof. As one example, one or more components of computer system 400 may be interconnected using compute express link (CXL) interconnects.

Inference and/or training logic 215 are used to perform inferencing and/or training operations associated with one or more embodiments. Details regarding inference and/or training logic 215 are provided below in conjunction with FIGS. 3B-3C. Inference and/or training logic 215 may be used in system FIG. 4 for inferencing or predicting operations based, at least in part, on weight parameters calculated using neural network training operations, neural network functions and/or architectures, or neural network use cases described herein.

Inference and/or training logic 215 are used to perform inferencing and/or training operations associated with one or more embodiments. This logic can be used with components of these figures to train one or more neural networks based, at least in part, on input data such as patient symptoms, states, or conditions and their corresponding diagnoses.

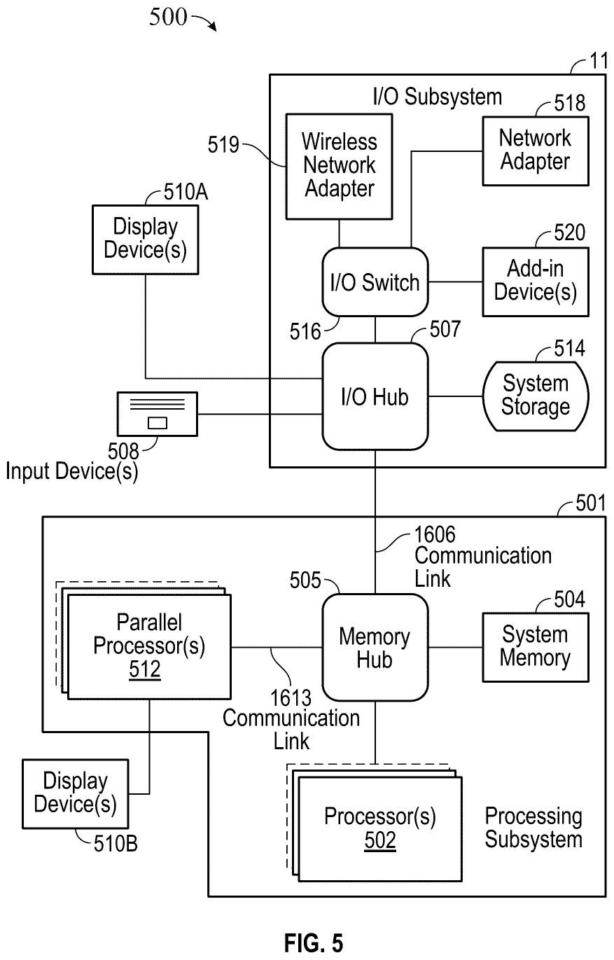

FIG. 5 illustrates a further exemplary computer system 500 for implementing a medical diagnosis system in accordance with various examples of the disclosure. Each computing device of FIG. 2 may be implemented as a computer system of FIG. 5. For example, diagnostics server 202, patient management server 204, usage and billing server 210, and doctor interface 212 may each be implemented on a computer system of FIG. 5. Computing system 500 includes a processing subsystem 501 having one or more processor(s) 502 and a system memory 504 communicating via an interconnection path that may include a memory hub 505. Memory hub 505 may be a separate component within a chipset component or may be integrated within one or more processor(s) 502. Memory hub 505 may couple with an I/O subsystem 511 via a communication link 506. I/O subsystem 511 includes an I/O hub 507 that can enable computing system 500 to receive input from one or more input device(s) 508. I/O hub 507 can enable a display controller, which may be included in one or more processor(s) 502, to provide outputs to one or more display device(s) 510A. One or more display device(s) 510A coupled with I/O hub 507 can include a local, internal, or embedded display device.

Processing subsystem 501 includes one or more parallel processor(s) 512 coupled to memory hub 505 via a bus or other communication link 513. Communication link 513 may be one of any number of standards based communication link technologies or protocols, such as, but not limited to PCI Express, or may be a vendor specific communications interface or communications fabric. One or more parallel processor(s) 512 form a computationally focused parallel or vector processing system that can include a large number of processing cores and/or processing clusters, such as a many integrated core (MIC) processor. One or more parallel processor(s) 512 can also include a display controller and display interface (not shown) to enable a direct connection to one or more display device(s) 510B.

A system storage unit 514 can connect to VO hub 507 to provide a storage mechanism for computing system 500. A VO switch 516 can be used to provide an interface mechanism to enable connections between I/O hub 507 and other components, such as a network adapter 518 and/or wireless network adapter 519 that may be integrated into a platform(s), and various other devices that can be added via one or more add-in device(s) 520. Network adapter 518 can be an Ethernet adapter or another wired network adapter. Wireless network adapter 519 can include one or more of a Wi-Fi, Bluetooth, near field communication (NFC), or other network device that includes one or more wireless radios.

Computing system 500 can include other components not explicitly shown, including USB or other port connections, optical storage drives, video capture devices, and like, may also be connected to VO hub 507. Communication paths interconnecting various components in FIG. 16 may be implemented using any suitable protocols, such as PCI (Peripheral Component Interconnect) based protocols (e.g., PCI-Express), or other bus or point-to-point communication interfaces and/or protocol(s), such as NV-Link high-speed interconnect, or interconnect protocols.

One or more parallel processor(s) 512 can incorporate circuitry optimized for any purpose, such as graphics and video processing, including, for example, video output circuitry, and can constitute a graphics processing unit (GPU). One or more parallel processor(s) 512 incorporate circuitry optimized for general purpose processing. Components of computing system 500 may be integrated with one or more other system elements on a single integrated circuit. For example, in at least one embodiment, one or more parallel processor(s) 512, memory hub 505, processor(s) 502, and VO hub 507 can be integrated into a system on chip (SoC) integrated circuit. Components of computing system 500 can be integrated into a single package to form a system in package (SIP) configuration. At least a portion of components of computing system 500 can be integrated into a multi-chip module (MCM), which can be interconnected with other multi-chip modules into a modular computing system.

In some embodiments, inference and/or training logic 215 may be used in system FIG. 500 for inferencing or predicting operations based, at least in part, on weight parameters calculated using neural network training operations, neural network functions and/or architectures, or neural network use cases described herein. Inference and/or training logic 215 are used to perform inferencing and/or training operations associated with one or more embodiments. This logic can be used with components of these figures to train one or more neural networks constructed according to embodiments of the disclosure, as described herein.

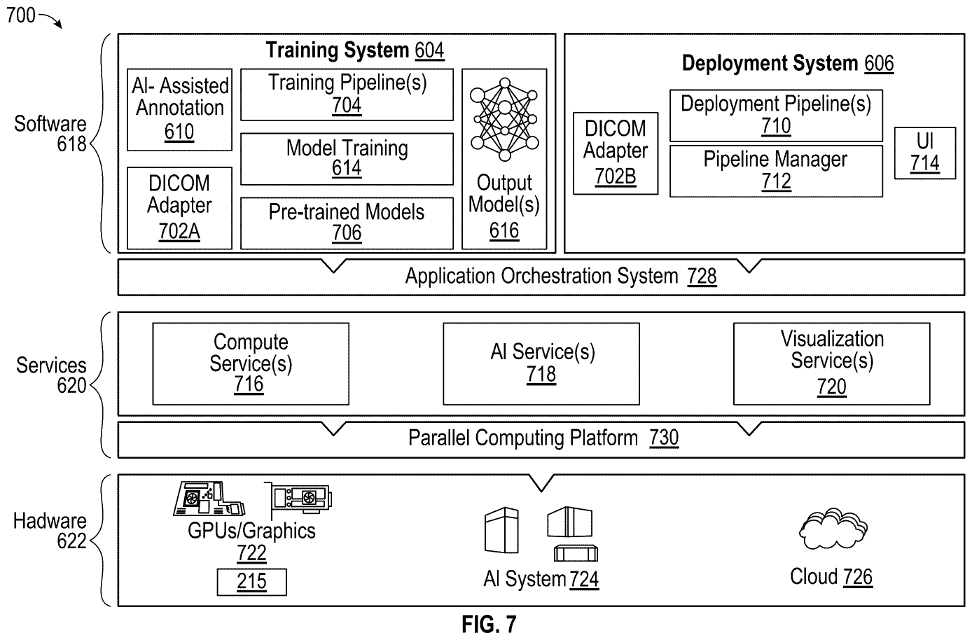

FIG. 6 illustrates data flow in an exemplary computing pipeline, in accordance with various examples of the disclosure, and FIG. 7 illustrates an exemplary system for training and deploying machine learning models in a computing system, in accordance with various examples of the disclosure.

FIG. 6 is an example data flow diagram for a process 600 of generating and deploying an input processing and inferencing pipeline, in accordance with at least one embodiment. Process 600 may be deployed for use with imaging devices, processing devices, data input devices, and/or other device types at one or more facilities 602, such as medical facilities, hospitals, healthcare institutes, clinics, research or diagnostic labs, etc. Process 600 may be deployed to perform symptom analysis and diagnosis inferencing on input patient data. Process 600 may be executed within a training system 604 and/or a deployment system 606. Training system 604 may be used to perform training, deployment, and implementation of machine learning models (e.g., neural networks, object detection algorithms, computer vision algorithms, etc.) for use in deployment system 606. Deployment system 606 may be configured to offload processing and compute resources among a distributed computing environment to reduce infrastructure requirements at facility 602. Deployment system 606 may provide a streamlined platform for selecting, customizing, and implementing virtual instruments for use with imaging devices (e.g., MRI, CT Scan, X-Ray, Ultrasound, etc.) or other data input devices at facility 602. Virtual instruments may include software-defined applications for performing one or more processing operations with respect to imaging data generated by imaging devices, sequencing devices, radiology devices, and/or other device types. One or more applications in a pipeline may use or call upon services (e.g., inference, visualization, compute, AI, etc.) of deployment system 606 during execution of applications.

Some of the applications used in advanced processing and inferencing pipelines may use machine learning models or other AI to perform one or more processing steps. Machine learning models may be trained at facility 602 using data 608 (such as input patient data) generated at facility 602 (and stored on one or more picture archiving and communication system (PACS) servers at facility 602), may be trained using imaging, sequencing, or other data 608 from another facility(ies) (e.g., a different hospital, lab, clinic, etc.), or a combination thereof. Training system 604 may be used to provide applications, services, and/or other resources for generating working, deployable machine learning models for deployment system 606.

Model registry 624 may be backed by object storage that may support versioning and object metadata. Object storage may be accessible through, for example, a cloud storage (e.g., cloud 726 of FIG. 7) compatible application programming interface (API) from within a cloud platform. Machine learning models within model registry 624 may be uploaded, listed, modified, or deleted by developers or partners of a system interacting with an API. An API may provide access to methods that allow users with appropriate credentials to associate models with applications, such that models may be executed as part of execution of containerized instantiations of applications.

Training pipeline 704 (FIG. 7) may include a scenario where facility 602 is training their own machine learning model, or has an existing machine learning model that needs to be optimized or updated. Here, imaging data 608 generated by imaging device(s), sequencing devices, and/or other data generated by other device types may be received. Once this imaging or other data 608 is received, Al-assisted annotation 610 may be used to aid in generating annotations corresponding to imaging data 608 to be used as ground truth data for a machine learning model. Al-assisted annotation 610 may include one or more machine learning models (e.g., convolutional neural networks (CNNs)) that may be trained to generate annotations corresponding to certain types of imaging data 608 (e.g., from certain devices) and/or certain types of anomalies in imaging data 608. Al-assisted annotations 610 may then be used directly, or may be adjusted or fine-tuned using an annotation tool (e.g., by a researcher, a clinician, a doctor, a scientist, etc.), to generate ground truth data. In some examples, labeled clinic data 612 (e.g., annotations provided by a clinician, doctor, scientist, technician, etc.) may be used as ground truth data for training a machine learning model. Al-assisted annotations 610, labeled clinic data 612, or a combination thereof may be used as ground truth data for training a machine learning model. A trained machine learning model may be referred to as output model 616, and may be used by deployment system 606, as described herein.

Training pipeline 704 (FIG. 7) may include a scenario where facility 602 needs a machine learning model for use in performing one or more processing tasks for one or more applications in deployment system 606, but facility 602 may not currently have such a machine learning model (or may not have a model that is optimized, efficient, or effective for such purposes). In that case, an existing machine learning model may be selected from a model registry 624. Model registry 624 may include machine learning models trained to perform a variety of different inference tasks on input data. For example, machine learning models in model registry 624 may have been trained on imaging data from different facilities than facility 602 (e.g., facilities remotely located), e.g., machine learning models may have been trained on imaging or other patient data from one location, two locations, or any number of locations. When being trained on imaging or other patient data from a specific location, training may take place at that location, or at least in a manner that protects confidentiality of imaging data or restricts imaging data from being transferred off-premises (e.g., to comply with HIPAA and/or GDPR regulations, privacy regulations, etc.). Once a model is trained-or partially trained-at one location, a machine learning model may be added to model registry 624. A machine learning model may then be retrained, or updated, at any number of other facilities, and a retrained or updated model may be made available in model registry 624. A machine learning model may then be selected from model registry 624—and referred to as output model 616—and may be used in deployment system 606 to perform one or more processing tasks for one or more applications of a deployment system.

In an exemplary training pipeline 704 (FIG. 7), a scenario may include facility 602 requiring a machine learning model for use in performing one or more processing tasks for one or more applications in deployment system 606, but facility 602 may not currently have such a machine learning model (or may not have a model that is optimized, efficient, or effective for such purposes). A machine learning model selected from model registry 624 may not be fine-tuned or optimized for imaging data 608 generated at facility 602 because of differences in populations, genetic variations, robustness of training data used to train a machine learning model, diversity in anomalies of training data, and/or other issues with training data. Al-assisted annotation 610 may be used to aid in generating annotations corresponding to imaging data 608 to be used as ground truth data for retraining or updating a machine learning model. Labeled clinic data 612 (e.g., annotations provided by a clinician, doctor, scientist, etc.) may be used as ground truth data for training a machine learning model. Retraining or updating a machine learning model may be referred to as model training 614. Model training 614—e.g., Al-assisted annotations 610, labeled clinic data 612, or a combination thereof—may be used as ground truth data for retraining or updating a machine learning model. A trained machine learning model may be referred to as output model 616, and may be used by deployment system 606, as described herein.

Deployment system 606 may include software 618, services 620, hardware 622, and/or other components, features, and functionality. Deployment system 606 may include a software “stack,” such that software 618 may be built on top of services 620 and may use services 620 to perform some or all of processing tasks, and services 620 and software 618 may be built on top of hardware 622 and use hardware 622 to execute processing, storage, and/or other compute tasks of deployment system 606. Software 618 may include any number of different containers, where each container may execute an instantiation of an application. Each application may perform one or more processing tasks in an advanced processing and inferencing pipeline (e.g., inferencing, object detection, feature detection, segmentation, image enhancement, calibration, etc.). For each type of imaging device (e.g., CT, MRI, X-Ray, ultrasound, sonography, echocardiography, etc.), sequencing device, radiology device, genomics device, etc., there may be any number of containers that may perform a data processing task with respect to imaging data 608 (or other data types, such as those described herein) generated by a device. An advanced processing and inferencing pipeline may be defined based on selections of different containers that are desired or required for processing imaging data 608, in addition to containers that receive and configure imaging or other data for use by each container and/or for use by facility 602 after processing through a pipeline (e.g., to convert outputs back to a usable data type, such as digital imaging and communications in medicine (DICOM) data, radiology information system (RIS) data, clinical information system (CIS) data, remote procedure call (RPC) data, data substantially compliant with a representation state transfer (REST) interface, data substantially compliant with a file-based interface, and/or raw data, for storage and display at facility 602). A combination of containers within software 618 (e.g., that make up a pipeline) may be referred to as a virtual instrument (as described in more detail herein), and a virtual instrument may leverage services 620 and hardware 622 to execute some or all processing tasks of applications instantiated in containers.

A data processing pipeline may receive input data (e.g., imaging data 608 or another data type) in a DICOM, RIS, CIS, REST compliant, RPC, raw, and/or other format in response to an inference request (e.g., a request from a user of deployment system 606, such as a clinician, a doctor, a radiologist, etc.). Input data may be representative of one or more images, video, and/or other data representations generated by one or more imaging devices, sequencing devices, radiology devices, genomics devices, and/or other device types, including patient 100 information entered by MF 102. Data may undergo pre-processing as part of data processing pipeline to prepare data for processing by one or more applications. Post-processing may be performed on an output of one or more inferencing tasks or other processing tasks of a pipeline to prepare an output data for a next application and/or to prepare output data for transmission and/or use by a user (e.g., as a response to an inference request). Inferencing tasks may be performed by one or more machine learning models, such as trained or deployed neural networks, which may include output models 616 of training system 604.

Tasks of data processing pipeline may be encapsulated in containers that each represent a discrete, fully functional instantiation of an application and virtualized computing environment that is able to reference machine learning models. For example, containers or applications may be published into a private (e.g., limited access) area of a container registry (described in more detail herein), and trained or deployed models may be stored in model registry 624 and associated with one or more applications. Images of applications (e.g., container images) may be available in a container registry, and once selected by a user from a container registry for deployment in a pipeline, an image may be used to generate a container for an instantiation of an application for use by a user's system.

Developers (e.g., software developers, clinicians, doctors, etc.) may develop, publish, and store applications (e.g., as containers) for performing image processing and/or inferencing on supplied data. Development, publishing, and/or storing may be performed using a software development kit (SDK) associated with a system (e.g., to ensure that an application and/or container developed is compliant with or compatible with a system). An application that is developed may be tested locally (e.g., at a first facility, on data from a first facility) with an SDK which may support at least some of services 620 as a system (e.g., system 700 of FIG. 7). Because DICOM objects may contain anywhere from one to hundreds of images or other data types, and due to a variation in data, a developer may be responsible for managing (e.g., setting constructs for, building pre-processing into an application, etc.) extraction and preparation of incoming DICOM data. Once validated by system 700 (e.g., for accuracy, safety, patient privacy, etc.), an application may be available in a container registry for selection and/or implementation by a user (e.g., a hospital, clinic, lab, healthcare provider, etc.) to perform one or more processing tasks with respect to data at a facility (e.g., a second facility) of a user.

Developers may then share applications or containers through a network for access and use by users of a system (e.g., system 700 of FIG. 7). Completed and validated applications or containers may be stored in a container registry and associated machine learning models may be stored in model registry 624. A requesting entity (e.g., a user at a medical facility)—who provides an inference or image processing request—may browse a container registry and/or model registry 624 for an application, container, dataset, machine learning model, etc., select a desired combination of elements for inclusion in data processing pipeline, and submit an imaging processing request. A request may include input data (and associated patient data, in some examples) that is necessary to perform a request, and/or may include a selection of application(s) and/or machine learning models to be executed in processing a request. A request may then be passed to one or more components of deployment system 606 (e.g., a cloud) to perform processing of data processing pipeline. Processing by deployment system 606 may include referencing selected elements (e.g., applications, containers, models, etc.) from a container registry and/or model registry 624. Once results are generated by a pipeline, results may be returned to a user for reference (e.g., for viewing in a viewing application suite executing on a local, on-premises workstation or terminal). For example, a doctor may receive results from an data processing pipeline including any number of applications and/or containers, where results may include various diagnoses, differential diagnoses, relevant patient information, etc.

To aid in processing or execution of applications or containers in pipelines, services 620 may be leveraged. Exemplary services 620 may include compute services, artificial intelligence (Al) services, visualization services, and/or other service types. Services 620 may provide functionality that is common to one or more applications in software 618, so functionality may be abstracted to a service that may be called upon or leveraged by applications. Functionality provided by services 620 may run dynamically and more efficiently, while also scaling well by allowing applications to process data in parallel (e.g., using a parallel computing platform 730 (FIG. 7)). In some embodiments, rather than each application that shares a same functionality offered by a service 620 being required to have a respective instance of service 620, service 620 may be shared between and among various applications. Services may include an inference server or engine that may be used for executing detection or segmentation tasks, as non-limiting examples. A model training service may be included that may provide machine learning model training and/or retraining capabilities. A data augmentation service may further be included that may provide GPU accelerated data (e.g., DICOM, RIS, CIS, REST compliant, RPC, raw, etc.) extraction, resizing, scaling, and/or other augmentation. A visualization service may be used that may add image rendering effects—such as ray-tracing, rasterization, denoising, sharpening, etc.—to add realism to two-dimensional (2D) and/or three-dimensional (3D) models. Virtual instrument services may be included that provide for beam-forming, segmentation, inferencing, imaging, and/or support for other applications within pipelines of virtual instruments.

Where a service 620 includes an AI service (e.g., an inference service), one or more machine learning models associated with an application for anomaly detection (e.g., tumors, growth abnormalities, scarring, etc.) may be executed by calling upon (e.g., as an API call) an inference service (e.g., an inference server) to execute machine learning model(s), or processing thereof, as part of application execution. Where another application includes one or more machine learning models for segmentation tasks, an application may call upon an inference service to execute machine learning models for performing one or more of processing operations associated with segmentation tasks. A software 618 implementing advanced processing and inferencing pipeline that includes a segmentation application and an anomaly detection application may be streamlined because each application may call upon a same inference service to perform one or more inferencing tasks.

Hardware 622 may include GPUs, CPUs, graphics cards, an Al/deep learning system (e.g., an AI supercomputer, such as NVIDIA's DGX), a cloud platform, or a combination thereof. Different types of hardware 622 may be used to provide efficient, purpose-built support for software 618 and services 620 in deployment system 606. Use of GPU processing may be implemented for processing locally (e.g., at facility 602), within an Al/deep learning system, in a cloud system, and/or in other processing components of deployment system 606 to improve efficiency, accuracy, and efficacy of image processing, image reconstruction, segmentation, MRI exams, stroke or heart attack detection (e.g., in real-time), image quality in rendering, etc. A facility may include imaging devices, genomics devices, sequencing devices, and/or other device types on-premises that may leverage GPUs to generate imaging data representative of a subject's anatomy. Software 618 and/or services 620 may be optimized for GPU processing with respect to deep learning, machine learning, and/or high-performance computing, as non-limiting examples. Datacenters may be compliant with provisions of HIPAA and/or GDPR, such that receipt, processing, and transmission of imaging data and/or other patient data is securely handled with respect to privacy of patient data. Hardware 622 may include any number of GPUs that may be called upon to perform processing of data in parallel, as described herein. Cloud platforms may further include GPU processing for GPU-optimized execution of deep learning tasks, machine learning tasks, or other computing tasks. As one example, a cloud platform of embodiments of the disclosure (e.g., NVIDIA's NGC) may be executed using an Al/deep learning computers and/or GPU-optimized software (e.g., as provided on NVIDIA′ s DGX Systems) as a hardware abstraction and scaling platform. A cloud platform of embodiments of the disclosure may integrate an application container clustering system or orchestration system (e.g., KUBERNETES) on multiple GPUs to enable seamless scaling and load balancing.

FIG. 7 is a system diagram for an example system 700 for generating and deploying a model deployment pipeline, in accordance with at least one embodiment. System 700 may be used to implement process 600 of FIG. 6 and/or other processes including advanced processing and inferencing pipelines. System 700 may include training system 604 and deployment system 606. Training system 604 and deployment system 606 may be implemented using software 618, services 620, and/or hardware 622, as described herein.

System 700 (e.g., training system 604 and/or deployment system 606) may implemented in a cloud computing environment (e.g., using cloud 726). System 700 may be implemented locally with respect to a healthcare services facility, or as a combination of both cloud and local computing resources. In embodiments where cloud computing is implemented, patient data may be separated from, or unprocessed by, by one or more components of system 700 that would render processing non-compliant with HIPAA, GDPR, and/or other data handling and privacy regulations or laws. Access to APIs in cloud 726 may be restricted to authorized users through enacted security measures or protocols. A security protocol may include web tokens that may be signed by an authentication (e.g., AuthN, AuthZ, Gluecon, etc.) service and may carry appropriate authorization. APIs of virtual instruments (described herein), or other instantiations of system 700, may be restricted to a set of public IPs that have been vetted or authorized for interaction.

Various components of system 700 may communicate between and among one another using any of a variety of different network types, including but not limited to local area networks (LANs) and/or wide area networks (WANs) via wired and/or wireless communication protocols. Communication between facilities and components of system 700 (e.g., for transmitting inference requests, for receiving results of inference requests, etc.) may be communicated over data bus(es), wireless data protocols (Wi-Fi), wired data protocols (e.g., Ethernet), etc.

Training system 604 may execute training pipelines 704 similar to those described herein with respect to FIG. 6. Where one or more machine learning models are to be used in deployment pipelines 710 by deployment system 606, training pipelines 704 may be used to train or retrain one or more (e.g. pretrained) models, and/or implement one or more of pre-trained models 706 (e.g., without a need for retraining or updating). As a result of training pipelines 704, output model(s) 616 may be generated. Training pipelines 704 may include any number of processing steps, such as but not limited to imaging data (or other input data) conversion or adaption (e.g., using DICOM adapter 702A to convert DICOM images to another format suitable for processing by respective machine learning models, such as Neuroimaging Informatics Technology Initiative (Nlf I) format), Al-assisted annotation 610, labeling or annotating of imaging data 608 to generate labeled clinic data 612, model selection from a model registry, model training 614, training, retraining, or updating models, and/or other processing steps. For different machine learning models used by deployment system 606, different training pipelines 704 may be used. For example, a training pipeline 704 similar to a first example described with respect to FIG. 6 may be used for a first machine learning model, a training pipeline 704 similar to a second example described with respect to FIG. 6 may be used for a second machine learning model, and a training pipeline 704 similar to a third example described with respect to FIG. 6 may be used for a third machine learning model. Any combination of tasks within training system 604 may be used depending on what is required for each respective machine learning model. One or more of machine learning models may already be trained and ready for deployment so machine learning models may not undergo any processing by training system 604, and may be implemented by deployment system 606.

Output model(s) 616 and/or pre-trained model(s) 706 may include any types of machine learning models used by embodiments of the disclosure depending on implementation or embodiment. Without limitation, machine learning models used by system 700 may include machine learning model(s) using linear regression, logistic regression, decision trees, support vector machines (SVM), Naive Bayes, k-nearest neighbor (Knn), K means clustering, random forest, dimensionality reduction algorithms, gradient boosting algorithms, neural networks (e.g., auto-encoders, convolutional, recurrent, perceptrons, Long/Short Term Memory (LSTM), Hopfield, Boltzmann, deep belief, deconvolutional, generative adversarial, liquid state machine, etc.), transformers, and/or other types of machine learning models.

In at least one embodiment, labeled clinical data 612 (e.g., traditional annotation) may be generated by any number of techniques. As an example, labels or other annotations may be generated within a drawing program (e.g., an annotation program), a computer aided design (CAD) program, a labeling program, another type of program suitable for generating annotations or labels for ground truth, and/or may be hand drawn, in some examples. Ground truth data may be synthetically produced (e.g., generated from computer models or renderings), real produced (e.g., designed and produced from real-world data), machine-automated (e.g., using feature analysis and learning to extract features from data and then generate labels), human annotated (e.g., labeler, or annotation expert, defines location of labels), and/or a combination thereof. For each instance of imaging data 608 (or other data type used by machine learning models), there may be corresponding ground truth data generated by training system 604. Al-assisted annotation may be performed as part of deployment pipelines 710; either in addition to, or in lieu of Al-assisted annotation included in training pipelines 704. System 700 may include a multi-layer platform that may include a software layer (e.g., software 618) of diagnostic applications (or other application types) that may perform one or more medical imaging and diagnostic functions. System 700 may be communicatively coupled to (e.g., via encrypted links) PACS server networks of one or more facilities. System 700 may be configured to access and referenced data (e.g., DICOM data, RIS data, raw data, CIS data, REST compliant data, RPC data, raw data, etc.) from PACS servers (e.g., via a DICOM adapter 702, or another data type adapter such as RIS, CIS, REST compliant, RPC, raw, etc.) to perform operations, such as training machine learning models, deploying machine learning models, image processing, inferencing, and/or other operations.

A software layer may be implemented as a secure, encrypted, and/or authenticated API through which applications or containers may be invoked (e.g., called) from an external environment(s) (e.g., facility 602). Applications may then call or execute one or more services 620 for performing compute, AI, or visualization tasks associated with respective applications, and software 618 and/or services 620 may leverage hardware 622 to perform processing tasks in an effective and efficient manner.

Deployment system 606 may execute deployment pipelines 710. In at least one embodiment, deployment pipelines 710 may include any number of applications that may be sequentially, non-sequentially, or otherwise applied to imaging data (and/or other data types) generated by imaging devices, sequencing devices, genomics devices, etc.—including Al-assisted annotation, as described above. In at least one embodiment, as described herein, a deployment pipeline 710 for an individual device may be referred to as a virtual instrument for a device (e.g., a virtual ultrasound instrument, a virtual CT scan instrument, a virtual sequencing instrument, etc.). In at least one embodiment, for a single device, there may be more than one deployment pipeline 710 depending on information desired from data generated by a device. In at least one embodiment, where detections of anomalies are desired from an MRI machine, there may be a first deployment pipeline 710, and where image enhancement is desired from output of an MRI machine, there may be a second deployment pipeline 710.

In at least one embodiment, applications available for deployment pipelines 710 may include any application that may be used for performing processing tasks on patient data or other data from devices. In at least one embodiment, different applications may be responsible for image enhancement, segmentation, reconstruction, anomaly detection, object detection, feature detection, treatment planning, dosimetry, beam planning (or other radiation treatment procedures), and/or other analysis, image processing, or inferencing tasks. In at least one embodiment, deployment system 606 may define constructs for each of applications, such that users of deployment system 606 (e.g., medical facilities, labs, clinics, etc.) may understand constructs and adapt applications for implementation within their respective facility. In at least one embodiment, an application for image reconstruction may be selected for inclusion in deployment pipeline 710, but data type generated by an imaging device may be different from a data type used within an application. In at least one embodiment, DICOM adapter 702B (and/or a DICOM reader) or another data type adapter or reader (e.g., RIS, CIS, REST compliant, RPC, raw, etc.) may be used within deployment pipeline 710 to convert data to a form useable by an application within deployment system 606. In at least one embodiment, access to DICOM, RIS, CIS, REST compliant, RPC, raw, and/or other data type libraries may be accumulated and pre-processed, including decoding, extracting, and/or performing any convolutions, color corrections, sharpness, gamma, and/or other augmentations to data. In at least one embodiment, DICOM, RIS, CIS, REST compliant, RPC, and/or raw data may be unordered and a pre-pass may be executed to organize or sort collected data. In at least one embodiment, because various applications may share common image operations, in some embodiments, a data augmentation library (e.g., as one of services 620) may be used to accelerate these operations. In at least one embodiment, to avoid bottlenecks of conventional processing approaches that rely on CPU processing, parallel computing platform 730 may be used for GPU acceleration of these processing tasks.

AI services 718 may be leveraged to perform inferencing services for executing machine learning model(s) associated with applications (e.g., tasked with performing one or more processing tasks of an application). AI services 718 may leverage AI system 724 to execute machine learning model(s) (e.g., neural networks, such as CNNs) for segmentation, reconstruction, object detection, feature detection, classification, and/or other inferencing tasks. Applications of deployment pipeline(s) 710 may use one or more of output models 616 from training system 604 and/or other models of applications to perform inference on imaging data (e.g., DICOM data, RIS data, CIS data, REST compliant data, RPC data, raw data, etc.). In some embodiments, two or more examples of inferencing using application orchestration system 728 (e.g., a scheduler) may be available. A first category may include a high priority/low latency path that may achieve higher service level agreements, such as for performing inference on urgent requests during an emergency, or for a radiologist during diagnosis. A second category may include a standard priority path that may be used for requests that may be non-urgent or where analysis may be performed at a later time. Application orchestration system 728 may distribute resources (e.g., services 620 and/or hardware 622) based on priority paths for different inferencing tasks of AI services 718.

Shared storage may be mounted to AI services 718 within system 700. Shared storage may operate as a cache (or other storage device type) and may be used to process inference requests from applications. When an inference request is submitted, a request may be received by a set of API instances of deployment system 606, and one or more instances may be selected (e.g., for best fit, for load balancing, etc.) to process a request. To process a request, a request may be entered into a database, a machine learning model may be located from model registry 624 if not already in a cache, a validation step may ensure appropriate machine learning model is loaded into a cache (e.g., shared storage), and/or a copy of a model may be saved to a cache. A scheduler (e.g., of pipeline manager 712) may be used to launch an application that is referenced in a request if an application is not already running or if there are not enough instances of an application. If an inference server is not already launched to execute a model, an inference server may be launched. Any number of inference servers may be launched per model. In a pull model, in which inference servers are clustered, models may be cached whenever load balancing is advantageous. Inference servers may be statically loaded in corresponding, distributed servers.

In some embodiments, inferencing may be performed using an inference server that runs in a container. An instance of an inference server may be associated with a model (and optionally a plurality of versions of a model). If an instance of an inference server does not exist when a request to perform inference on a model is received, a new instance may be loaded. When starting an inference server, a model may be passed to an inference server such that a same container may be used to serve different models so long as inference server is running as a different instance.

During application execution, an inference request for a given application may be received, and a container (e.g., hosting an instance of an inference server) may be loaded (if not already), and a start procedure may be called. Pre-processing logic in a container may load, decode, and/or perform any additional pre-processing on incoming data (e.g., using a CPU(s) and/or GPU(s)). Once data is prepared for inference, a container may perform inference as necessary on data. This may include a single inference call on one image (e.g., a hand X-ray) or other data, or may require inference on hundreds of images (e.g., a chest CT) or collections of input data. An application may summarize results before completing, which may include, without limitation, a single confidence score, pixel level-segmentation, voxel-level segmentation, generating a visualization, or generating text to summarize findings. Different models or applications may be assigned different priorities. For example, some models may have a real-time (TAT<1 min) priority while others may have lower priority (e.g., TAT<10 min). Model execution times may be measured from requesting institution or entity and may include partner network traversal time, as well as execution on an inference service.

Visualization services 720 may be leveraged to generate visualizations for viewing outputs of applications and/or deployment pipeline(s) 710. GPUs 722 may be leveraged by visualization services 720 to generate visualizations. Rendering effects, such as ray-tracing, may be implemented by visualization services 720 to generate higher quality visualizations. Visualizations may include, without limitation, 2D image renderings, 3D volume renderings, 3D volume reconstruction, 2D tomographic slices, virtual reality displays, augmented reality displays, etc. Virtualized environments may be used to generate a virtual interactive display or environment (e.g., a virtual environment) for interaction by users of a system (e.g., doctors, nurses, radiologists, etc.). Visualization services 720 may include an internal visualizer, cinematics, and/or other rendering or image processing capabilities or functionality (e.g., ray tracing, rasterization, internal optics, etc.).

Hardware 622 may include GPUs 722, AI system 724, cloud 726, and/or any other hardware used for executing training system 604 and/or deployment system 606. GPUs 722 (e.g., NVIDIA's TESLA and/or QUADRO GPUs) may include any number of GPUs that may be used for executing processing tasks of compute services 716, AI services 718, visualization services 720, other services, and/or any of features or functionality of software 618. For example, with respect to AI services 718, GPUs 722 may be used to perform pre-processing on imaging data (or other data types used by machine learning models), post-processing on outputs of machine learning models, and/or to perform inferencing (e.g., to execute machine learning models). Cloud 726, AI system 724, and/or other components of system 700 may use GPUs 722. Cloud 726 may include a GPU-optimized platform for deep learning tasks. AI system 724 may use GPUs, and cloud 726—or at least a portion tasked with deep learning or inferencing—may be executed using one or more AI systems 724. As such, although hardware 622 is illustrated as discrete components, this is not intended to be limiting, and any components of hardware 622 may be combined with, or leveraged by, any other components of hardware 622.

AI system 724 (e.g., NVIDIA's DGX) may include GPU-optimized software (e.g., a software stack) that may be executed using a plurality of GPUs 722, in addition to CPUs, RAM, storage, and/or other components, features, or functionality. One or more AI systems 724 may be implemented in cloud 726 (e.g., in a data center) for performing some or all of AI-based processing tasks of system 700.

Cloud 726 may include a GPU-accelerated infrastructure (e.g., NVIDIA's NGC) that may provide a GPU-optimized platform for executing processing tasks of system 700. Cloud 726 may include an AI system(s) 724 for performing one or more of Al-based tasks of system 700 (e.g., as a hardware abstraction and scaling platform). Cloud 726 may integrate with application orchestration system 728 leveraging multiple GPUs to enable seamless scaling and load balancing between and among applications and services 620. Cloud 726 may tasked with executing at least some of services 620 of system 700, including compute services 716, AI services 718, and/or visualization services 720, as described herein. Cloud 726 may perform small and large batch inference (e.g., executing NVIDIA's TENSOR RT), provide an accelerated parallel computing API and platform 730 (e.g., NVIDIA's CUDA), execute application orchestration system 728 (e.g., KUBERNETES), provide a graphics rendering API and platform (e.g., for ray-tracing, 2D graphics, 3D graphics, and/or other rendering techniques to produce higher quality cinematics), and/or may provide other functionality for system 700.

In an effort to preserve patient confidentiality (e.g., where patient data or records are to be used off-premises), cloud 726 may include a registry-such as a deep learning container registry. A registry may store containers for instantiations of applications that may perform pre-processing, post-processing, or other processing tasks on patient data. Cloud 726 may receive data that includes patient data as well as sensor data in containers, perform requested processing for just sensor data in those containers, and then forward a resultant output and/or visualizations to appropriate parties and/or devices (e.g., on-premises medical devices used for visualization or diagnoses), all without having to extract, store, or otherwise access patient data. Confidentiality of patient data is preserved in compliance with HIPAA, GDPR, and/or other data regulations.

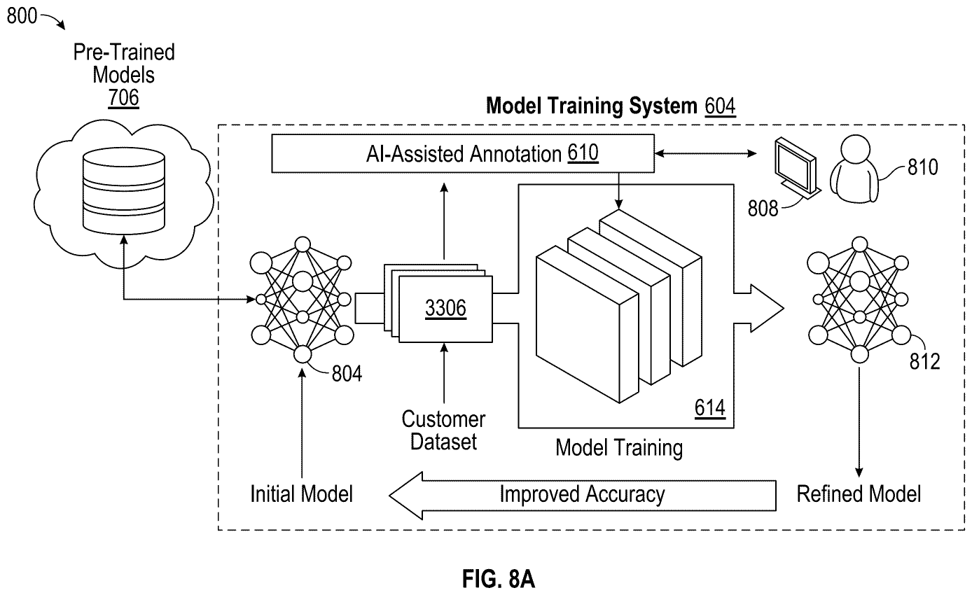

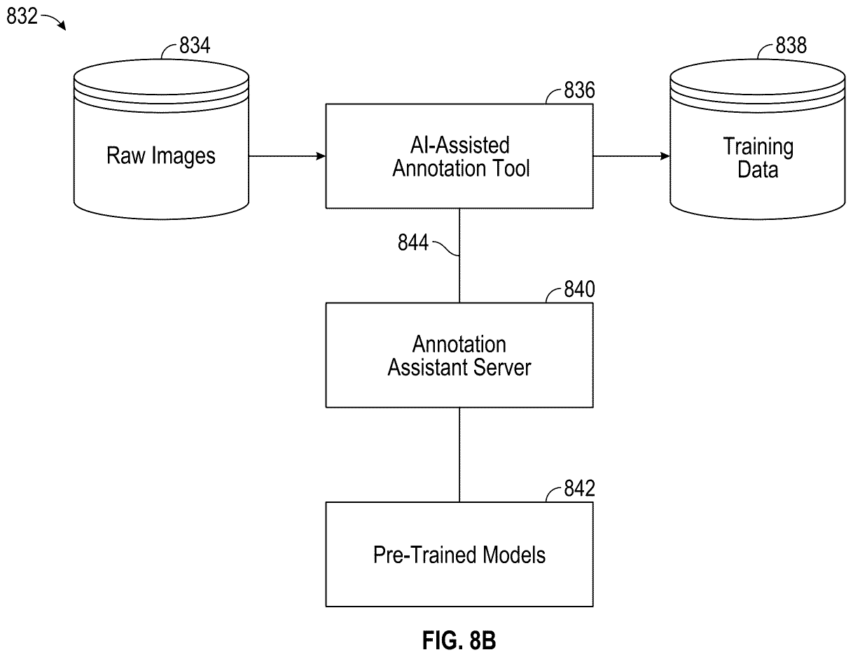

FIG. 8A illustrates a data flow diagram for a process 800 to train, retrain, or update a machine learning model, in accordance with at least one embodiment. Process 800 may be executed using, as a non-limiting example, system 700 of FIG. 7. Process 800 may leverage services 620 and/or hardware 622 of system 700, as described herein. Refined models 812 generated by process 800 may be executed by deployment system 3106 for one or more containerized applications in deployment pipelines 710.

Model training 614 may include retraining or updating an initial model 804 (e.g., a pre-trained model) using new training data (e.g., new input data, such as customer dataset 806, and/or new ground truth data associated with input data). To retrain, or update, initial model 804, output or loss layer(s) of initial model 804 may be reset, or deleted, and/or replaced with an updated or new output or loss layer(s). Initial model 804 may have previously fine-tuned parameters (e.g., weights and/or biases) that remain from prior training, so training or retraining 614 may not take as long or require as much processing as training a model from scratch. During model training 614, by having reset or replaced output or loss layer(s) of initial model 804, parameters may be updated and re-tuned for a new data set based on loss calculations associated with accuracy of output or loss layer(s) at generating predictions on new, customer dataset 806 (e.g., image data 608 of FIG. 6).

Pre-trained models 706 may be stored in a data store, or registry (e.g., model registry 624 of FIG. 6). Pre-trained models 706 may have been trained, at least in part, at one or more facilities other than a facility executing process 800. To protect privacy and rights of patients, subjects, or clients of different facilities, pre-trained models 706 may have been trained, on premise, using customer or patient data generated on-premise. Pretrained models 706 may be trained using cloud 726 and/or other hardware 622, but confidential, privacy protected patient data may not be transferred to, used by, or accessible to any components of cloud 726 (or other off premise hardware). Where a pre-trained model 706 is trained at using patient data from more than one facility, pretrained model 706 may have been individually trained for each facility prior to being trained on patient or customer data from another facility. In one example, such as where a customer or patient data has been released of privacy concerns (e.g., by waiver, for experimental use, etc.), or where a customer or patient data is included in a public data set, a customer or patient data from any number of facilities may be used to train pre-trained model 706 on premise and/or off premise, such as in a datacenter or other cloud computing infrastructure.

When selecting applications for use in deployment pipelines 710, a user may also select machine learning models to be used for specific applications. A user may not have a model for use, so a user may select a pre-trained model 706 to use with an application. Pre-trained model 706 may not be optimized for generating accurate results on customer dataset 806 of a facility of a user (e.g., based on patient diversity, demographics, types of medical imaging devices used, etc.). Prior to deploying pre-trained model 706 into deployment pipeline 710 for use with an application(s), pre-trained model 706 may be updated, retrained, and/or fine-tuned for use at a respective facility.

A user may select pre-trained model 706 that is to be updated, retrained, and/or fine-tuned, and pre-trained model 706 may be referred to as initial model 804 for training system 3104 within process 800. Customer dataset 806 (e.g., imaging data, genomics data, sequencing data, or other data types generated by devices at a facility) may be used to perform model training 614 (which may include, without limitation, transfer learning) on initial model 804 to generate refined model 812. Ground truth data corresponding to customer dataset 806 may be generated by training system 3104. In at least one embodiment, ground truth data may be generated, at least in part, by clinicians, scientists, doctors, practitioners, at a facility (e.g., as labeled clinic data 612 of FIG. 31).

Al-assisted annotation 610 may be used in some examples to generate ground truth data. Al-assisted annotation 610 (e.g., implemented using an Al-assisted annotation SDK) may leverage machine learning models (e.g., neural networks) to generate suggested or predicted ground truth data for a customer dataset. User 810 may use annotation tools within a user interface (a graphical user interface (GUI)) on computing device 808. For example, user 810 may interact with a GUI via computing device 808 to edit or fine-tune (auto)annotations. A polygon editing feature may be used to move vertices of a polygon to more accurate or fine-tuned locations.

Once customer dataset 806 has associated ground truth data, ground truth data (e.g., from Al-assisted annotation, manual labeling, etc.) may be used by during model training 614 to generate refined model 812. Customer dataset 806 may be applied to initial model 804 any number of times, and ground truth data may be used to update parameters of initial model 804 until an acceptable level of accuracy is attained for refined model 812. Once refined model 812 is generated, refined model 812 may be deployed within one or more deployment pipelines 710 at a facility for performing one or more processing tasks with respect to medical imaging data.

Refined model 812 may be uploaded to pre-trained models 706 in model registry 624 to be selected by another facility. This process may be completed at any number of facilities such that refined model 812 may be further refined on new datasets any number of times to generate a more universal model.

FIG. 8B is an example illustration of a client-server architecture 382 to enhance annotation tools with pre-trained annotation models, in accordance with at least one embodiment. Al-assisted annotation tools 836 may be instantiated based on a client-server architecture 382. Annotation tools 836 in imaging applications may aid radiologists, for example, identify organs and abnormalities. Imaging applications may include software tools that help user 810 to identify, as a non-limiting example, a few extreme points on a particular organ of interest in raw images 834 (e.g., in a 3D MRI or CT scan) and receive auto-annotated results for all 2D slices of a particular organ. Results may be stored in a data store as training data 838 and used as (for example and without limitation) ground truth data for training. When computing device 808 sends extreme points for Al-assisted annotation 610, a deep learning model, for example, may receive this data as input and return inference results of a segmented organ or abnormality. Preinstantiated annotation tools, such as AI-Assisted Annotation Tool 836B in FIG. 8B, may be enhanced by making API calls (e.g., API Call 844) to a server, such as an Annotation Assistant Server 840 that may include a set of pre-trained models 842 stored in an annotation model registry, for example. An annotation model registry may store pretrained models 842 (e.g., machine learning models, such as deep learning models) that are pretrained to perform Al-assisted annotation on a particular organ or abnormality. These models may be further updated by using training pipelines 704. Preinstalled annotation tools may be improved over time as new labeled clinic data 612 is added.

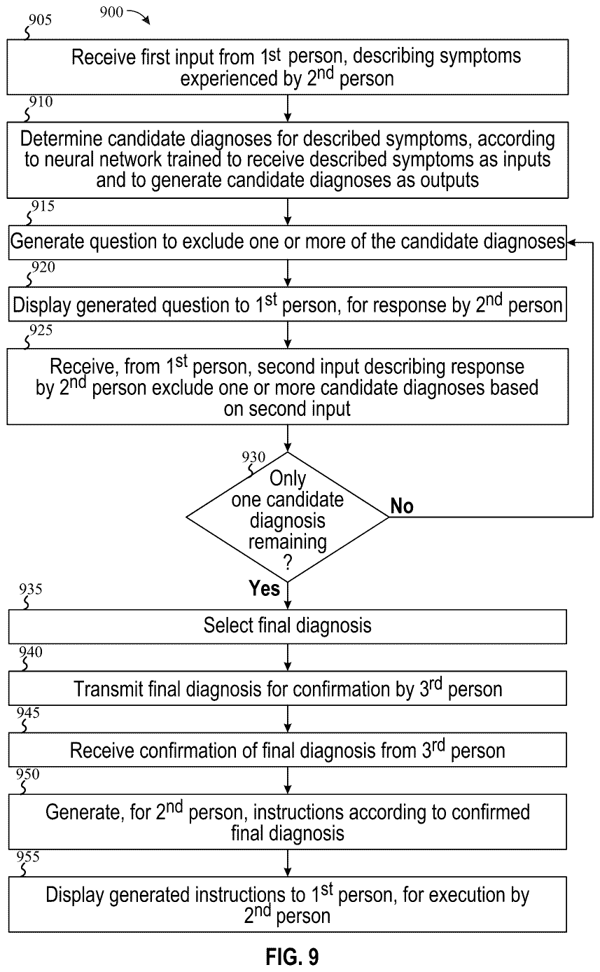

FIG. 9 is a flow chart representing a process for determining and providing medical diagnoses using a multi-person interface, in accordance with various examples of the disclosure. In embodiments of the disclosure, the process 900 of FIG. 9 may be carried out at least in part using an electronic device having one or more processors and a display. Process 900 may include receiving at an electronic device, from a first person such as an MF 102, a first user input describing symptoms experienced by a second person such as a patient 100 (Step 905). In some embodiments, and as above, a patient 100 experiencing symptoms may make an appointment with a medical facility for diagnosis and treatment. At the appointment, patient 100 may see an MF 102, who need not necessarily be a trained physician or medical doctor, and who serves as an intermediary “human link” between patients 100 and electronic medical applications that diagnose patients and deliver treatments. MF 102 may, for example, be a nurse or any other person trained to use the application programs of embodiments of the disclosure, and who has sufficient medical knowledge to follow the instructions of the application programs. In some embodiments, MF 102 is trained to query patient 100 for his or her symptoms, and to enter those symptoms to an electronic device executing application programs of embodiments of the disclosure, such as diagnostics system 104, patient management server 204, or the like. In some embodiments, MF 102 may take measurements of patient 100, such as vital signs or the like, via measurement devices 206, and enter them to diagnostics system 104 via MF interface 208.

Process 900 may next determine candidate diagnoses for the symptoms entered by MF 102 at Step 905, where the candidate diagnoses are determined by one or more neural networks that have been trained to receive the described symptoms as inputs and to generate the candidate diagnoses as outputs (Step 910). In some embodiments, a computing device such as patient management server 204 may execute one or more neural networks or other machine learning models which generate candidate diagnoses for the symptoms of patient 100 that it receives at Step 905. These machine learning models may be any machine learning models suitable for generating a medical diagnosis from an input set of patient symptoms. For example, the machine learning models executed by server 204 may include supervised machine learning models such as classifiers or regression models that may include any one or more of K-Nearest Neighbors (KNN) models, Bayesian models, random forest models, support vector machines, logistic models, gradient boosting models, or the like. Such classification and/or regression models may be trained to classify output diagnoses according to specified inputs, e.g., input symptoms and patient health data, in known manner. In some embodiments, classification models may predict categories or classes of diagnoses to which input symptoms may belong, while regression models may predict diagnoses as continuous variables based on input symptoms. Machine learning models may further include unsupervised machine learning models such as K-means or other clustering models, principal component analysis models, apriori models, singular value decomposition models, independent component analysis models, deep belief networks, recurrent neural networks including long short term memory networks, or the like. Machine learning models may further include any semi-supervised and/or reinforcement learning models such as self-training and co-training models, image classification models such as convolutional neural networks, and anomaly detection models. Machine learning models may also include one or more generative models, including without limitation generative adversarial networks, any transformer models including generative pre-trained transformers, and any language models including large language models (LLMs) trained on input text data to understand and generate readable text such as answers to questions or generated content, or the like. Embodiments of the disclosure contemplate any one or more machine learning models, constructed and arranged in any manner that may generate medical diagnoses from an input set of patient symptoms. These models may be trained by any suitable methods, such as the methods described above in connection with FIGS. 8A-8B. Labeled training data, if used, may comprise sets of symptoms and/or patient health information (e.g., vital signs, demographic data, patient health habits, and the like) labeled with corresponding diagnoses. Models of embodiments of the disclosure may be configured to generate any suitable outputs, including but not limited to probabilities of various diagnoses.

In some embodiments, one or more machine learning models may be employed to receive input audio/visual data generated by measurement devices 206, such as x-rays, MRI, ECG, or other images, voice inputs, transcribed text from audio output of patient 100, or any other media or content that may be useful in patient diagnosis. In some embodiments, machine learning models may be configured to perform diagnoses using input images, such as identifying cancerous growths in x-ray images or the like. In some embodiments, machine learning models may include generative models configured to generate diagnoses that may include content such as enhanced or clarified images (including video images) of diagnosed problem areas, or the like. Any content corresponding to diagnoses is contemplated, including without limitation still or video images highlighting or otherwise describing diagnosed conditions, and the like. In some embodiments, devices such as patient management server 204 may execute more than one machine learning model, where output of one model may be used as an input to a subsequent model. For example, an LLM may be configured to output transcriptions of patient 100 voice inputs, with these transcriptions serving as an input to a subsequent machine learning model that classifies speech disorders, detects strokes, or the like, according to inputs that include speech patterns. In embodiments including multiple machine learning models, each model may be individually trained or tuned for its specific task, including by training using data sets selected for the specific task for which each machine learning model is designated. Training data sets may thus include any data tailored to any specific task, including portions of EHR data, clinical notes, and the like relating to any specific patient symptoms or diagnoses.

In some embodiments, Step 910 may result in multiple candidate diagnoses. That is, machine learning models of embodiments of the disclosure may output multiple candidate diagnoses for a given set of input patient information. Accordingly, process 900 may next generate questions to exclude various ones of these multiple candidate diagnoses to, in some embodiments, result in a single diagnosis (Step 915). Any number of candidate diagnoses is contemplated, and any questions are contemplated to narrow the number of candidates down to any amount, e.g., one or more. For example, generated questions may include questions directed to excluding or confirming specific symptoms, such as questions directed to clarifying the nature of certain symptoms, questions determining when or how often certain symptoms occur, whether other symptoms occur along with the specific symptoms, or the like.

In some embodiments, questions may be generated and stored as a decision tree for each symptom or set of related symptoms. Such decision trees may be stored as data structures on, e.g., diagnostics server 202, where server 202 may retrieve and traverse specific decision trees corresponding to the candidate symptoms output by machine learning models at Step 910. Traversal of retrieved decision trees may thus generate or assist in generating questions. In some embodiments, questions may be automatically generated by one or more machine learning models trained to generate output questions from an input set of diagnoses.

Any suitable machine learning models are contemplated, including without limitation any of the models listed herein. As an example, classification and/or regression models may be trained to classify output questions according to specified inputs, e.g., input diagnoses and patient health data, in known manner. Labeled training data, if used, may comprise sets of diagnoses and/or patient health information (e.g., vital signs, demographic data, patient health habits, and the like) labeled with corresponding questions or information required for more accurate diagnosis.

Process 900 may next display the generated questions to the MF 102, for response by patient 100 (Step 920). Here, MF 102 may relay the displayed questions to patient 100, perhaps interpreting the displayed questions and relaying them to patient 100 in more readily understandable form, e.g., in their native language, with accompanying explanations of why such information may be required, or the like. Patient 100 responses may then be interpreted and entered into diagnostics system 104 by MF 102 (Step 925), to provide the diagnostics system 104 with information it may use to exclude one or more of the candidate diagnoses that were determined at Step 910. In some embodiments, above-described decision trees may include end nodes excluding certain diagnoses according to the answers received at Step 925. Embodiments of the disclosure contemplate any methods of excluding candidate diagnoses according to information from patients 100.

The process of Steps 915-925 may be repeated for each generated question as appropriate (Step 930), to successively exclude candidate diagnoses until only one, or only an acceptable number, remains. Once only one diagnosis remains, it is selected as the final diagnosis (Step 935). If multiple diagnoses are acceptable, they are each selected. Diagnostics server 202, diagnostics system 104, or the like may then transmit these final diagnoses to a qualified medical professional such as MD 108 for confirmation (Step 940). In some embodiments, the final diagnoses and any relevant supporting information such as the patient's medical record (retrieved from EHR 214) and symptoms are sent from diagnostics server 202 to doctor interface 212, whereupon MD 108 may review the information and either confirm the diagnosis, reject it, or request further information. Rejections or requests for further information may result in repetition of Steps 915-925 for a different diagnosis, an interview of patient 100 by MD 108 or MF 102, or the like. Any follow-up actions are contemplated. Step 940 serves as a review and confirmation step to ensure the accuracy and safety of the final diagnosis.

If the MD 108 confirms the final diagnosis, the confirmed final diagnosis is transmitted to the MF 102 (Step 945) for explanation to patient 100. Diagnostics server 202 may additionally generate applicable instructions for MF 102 to relay to patient 100 (Step 950), such as treatment methods suitable for the condition of the final diagnosis, information on the final diagnosis such as causes, mortality rates, treatment success likelihoods, or any other information that may be desired by patients 100. As above, information is generated for display to MF 102 for relaying to patient 100 (Step 955), rather than for display directly to patient 100. In this manner, MF 102 or another trained individual may relay the generated information in a manner more understandable and acceptable to patient 100, in the hope that he or she will be more likely to understand and follow the prescribed treatment.

In some embodiments, an appropriate treatment can include (but is not limited to) one or more of a pharmaceutical treatment (e.g., oral, injection, or topical medications), a surgical treatment, physical therapy treatment, curative treatment, palliative treatment, preventative treatment, behavioral therapy treatment, herbal treatment, and combinations thereof. Any treatment suitable for any diagnosis is contemplated. As above, treatments for each diagnosis may be predetermined and stored in a memory of any computing device of system 200 of embodiments of the disclosure. Alternatively, treatments may be determined by one or more machine learning models such as those described above, where the one or more models are trained to receive diagnoses and applicable patient 100 information as inputs, and to generate corresponding treatments as outputs. In some embodiments, treatments are displayed to MF 102 rather than directly to patient 100, so that MF 102 may perform all or part of the treatments on the patient 100 rather than relying on the patient 100 to treat themselves. Additionally, MF 102 may be trained to answer any follow-on questions patient 100 may have, to provide reassurance or support that an automated system cannot, or to simply relay the treatment or other diagnosis information generated by automated applications of embodiments of the disclosure in a human-to-human manner more readily acceptable by and digestible by patient 100. In this manner, embodiments of the disclosure provide a more flexible and understandable system than the rigid, inflexible, and often difficult to understand conventional direct-to-patient medical applications.

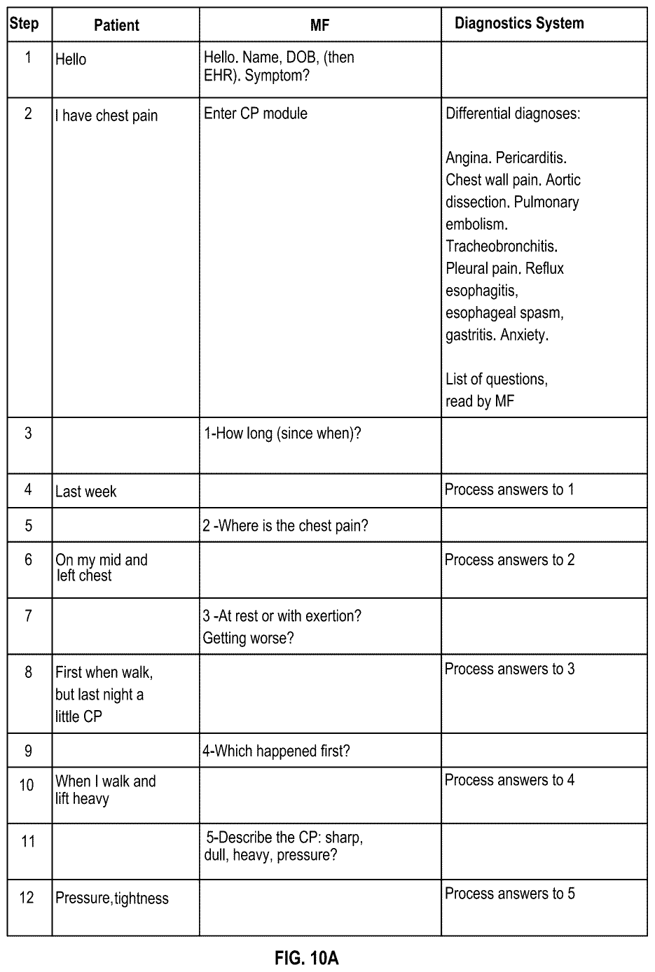

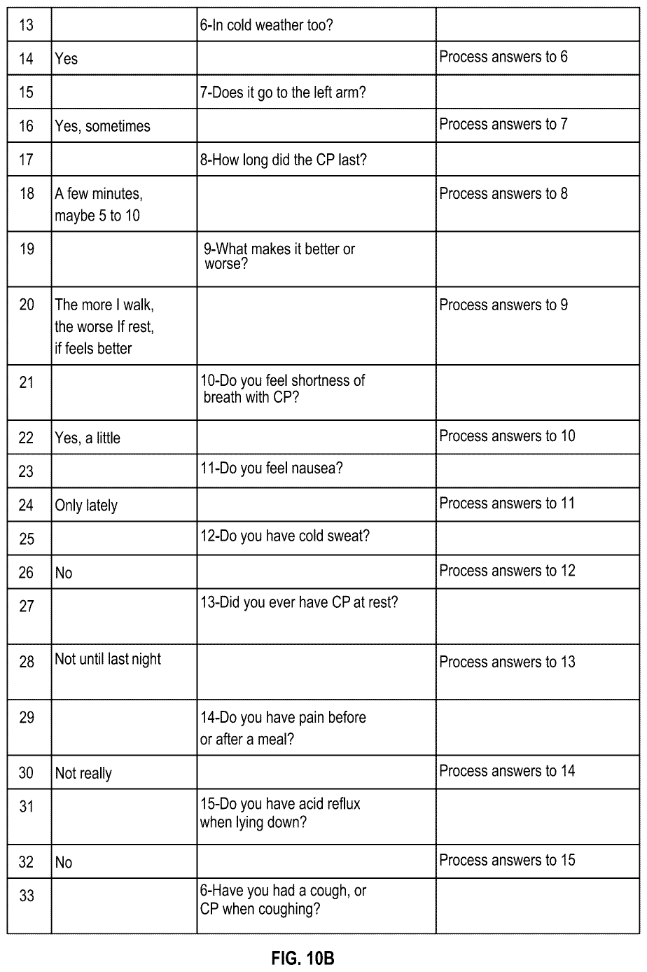

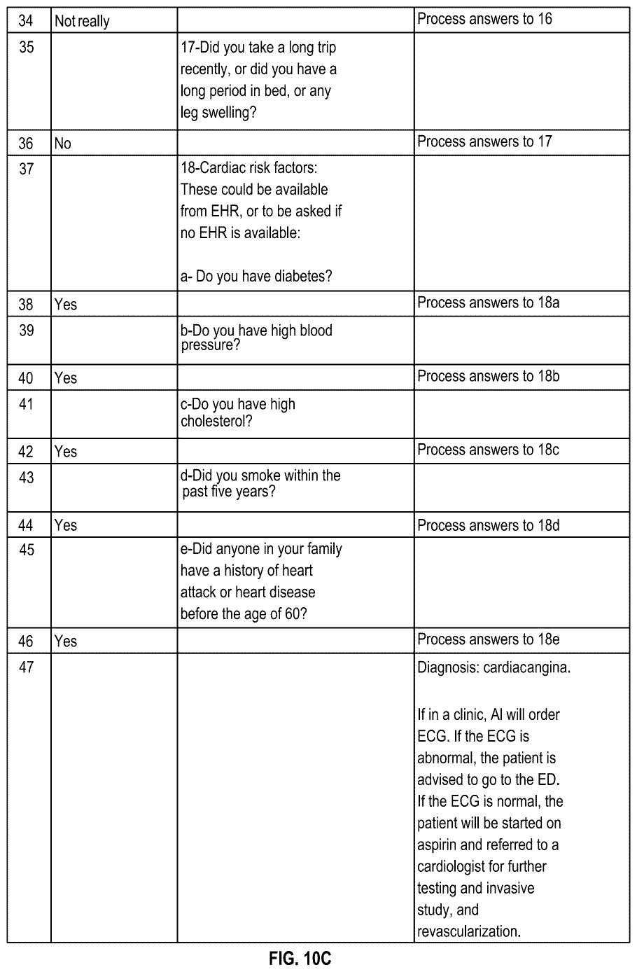

FIGS. 10A-10C are an exemplary illustration of a process for determining and providing medical diagnoses using a multi-person interface, in accordance with various examples of the disclosure. Here, the “MF” column of FIG. 10 lists questions generated by diagnostics system 104 as they are relayed to patient 100 by MF 102, while the “Patient” column of FIGS. 10A-10C lists exemplary answers that a patient 100 may give in response. Accordingly, at each listed step, FIGS. 10A-10C illustrate MF 102 questions (from diagnostics system 104) to patient 100, with the following step listing patient 100 answers. The steps also show the differential or candidate diagnoses generated by system 104 in response to the answers provided by patient 100. In this example, chest pain symptoms generate several different differential or potential diagnoses from system 104, i.e., the above described neural networks executed by, for example, diagnostics server 202. Also generated are a list of questions to be asked by MF 102, for exclusion of various differential diagnoses. The explanation or transmission of these questions by MF 102 to patient 100 is shown in the following several steps of FIGS. 10A-10C, with each question resulting in a patient 100 answer that is entered to diagnostics system 104 by MF 102. The end result of these questions and answers is a final diagnosis of cardiac angina, which may be determined by exclusion of the remaining differential or candidate diagnoses by the various answers given by patient 100. Treatment instructions are then listed as shown, for the MF 102 to relay to patient 100. For example, the MF 102 may be instructed to request an echocardiogram (ECG) for patient 100, along with a referral to a cardiologist for further testing/treatment, and immediate treatment with aspirin.

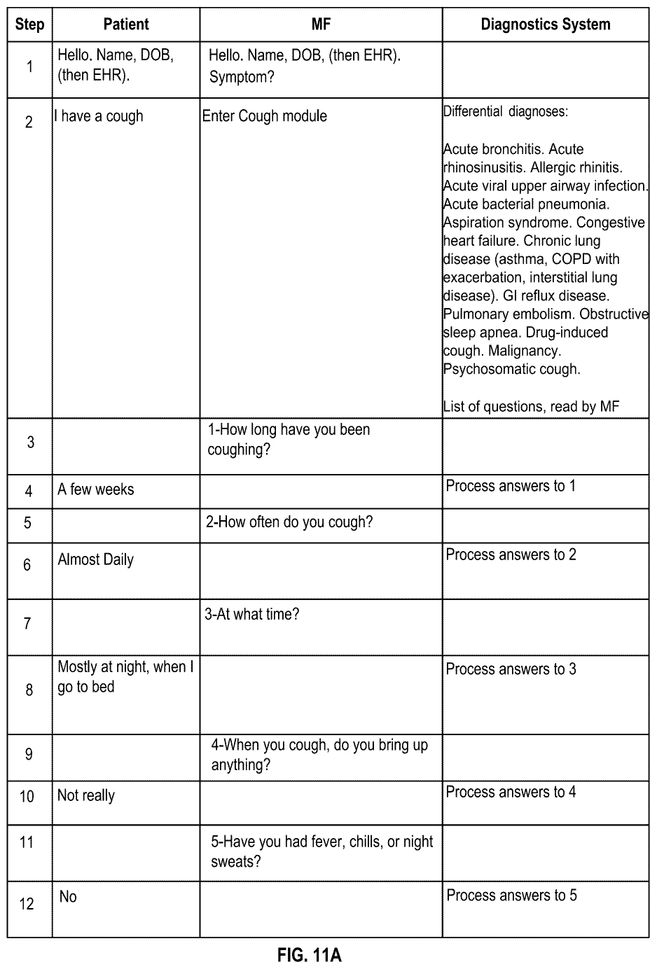

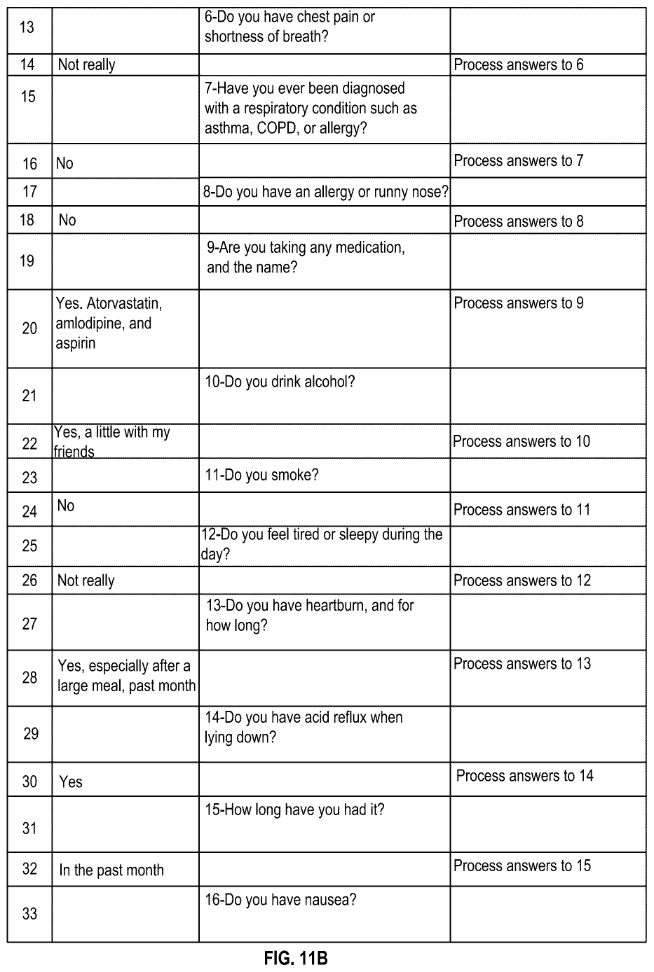

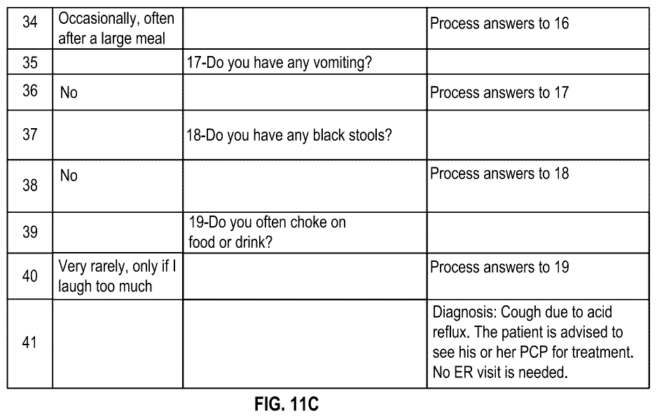

FIGS. 11A-11C are an exemplary illustration of a process for determining and providing a diagnosis of acid reflux using a multi-person interface, in accordance with various examples of the disclosure. Column names of FIGS. 11A-11C retain the same meanings as those of FIGS. 10A-10C. In this example, cough symptoms generate multiple differential or potential diagnoses generated by, e.g., the above described neural networks executed by, for example, diagnostics server 202. Also generated are a list of questions to be asked by MF 102, for exclusion of various differential diagnoses. The explanation or transmission of these questions by MF 102 to patient 100 is shown in the following several steps, with each question resulting in a patient 100 answer that is entered to diagnostics system 104 by MF 102. The end result of these questions and answers is a final diagnosis of cough due to acid reflux, which may be determined as in the previous example by exclusion of the remaining differential or candidate diagnoses by the various answers given by patient 100. Treatment instructions are then listed as shown, for the MF 102 to relay to patient 100. For example, the MF 102 may be instructed to see his or her primary care physician (PCP) for examination or treatment, with no emergency room (ER) visit needed.

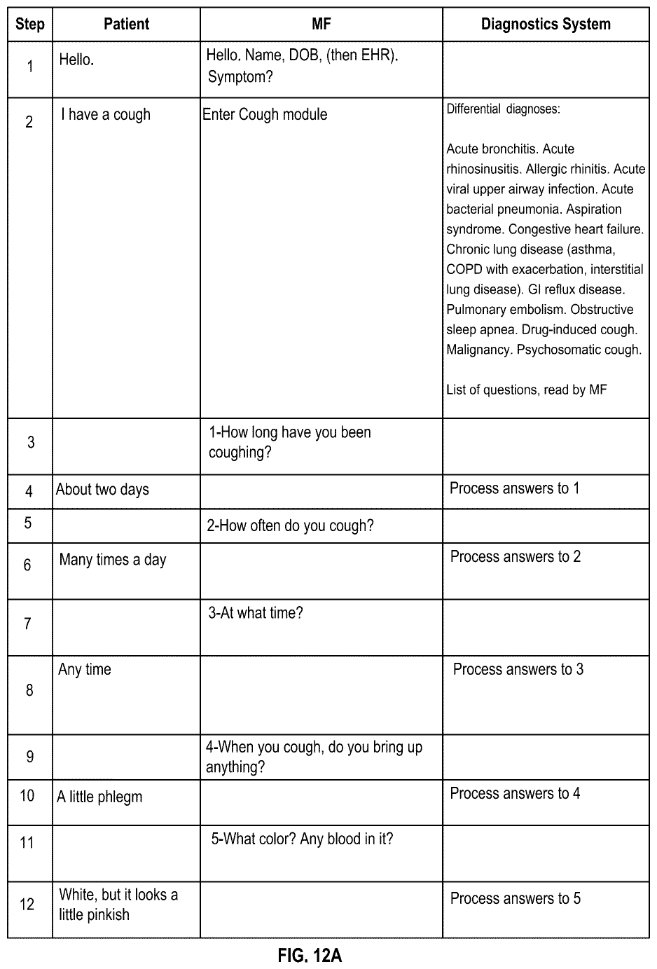

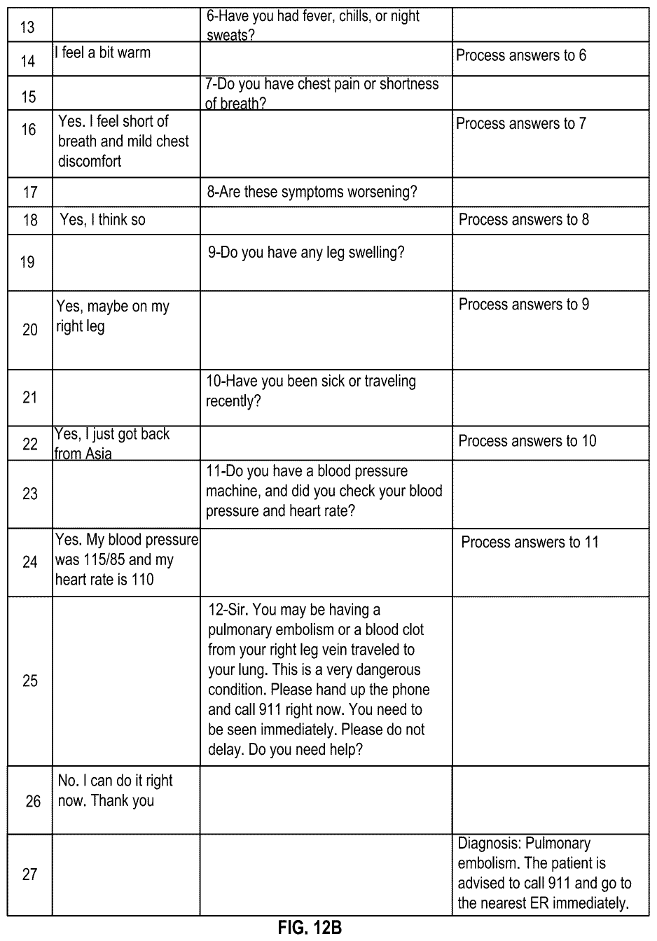

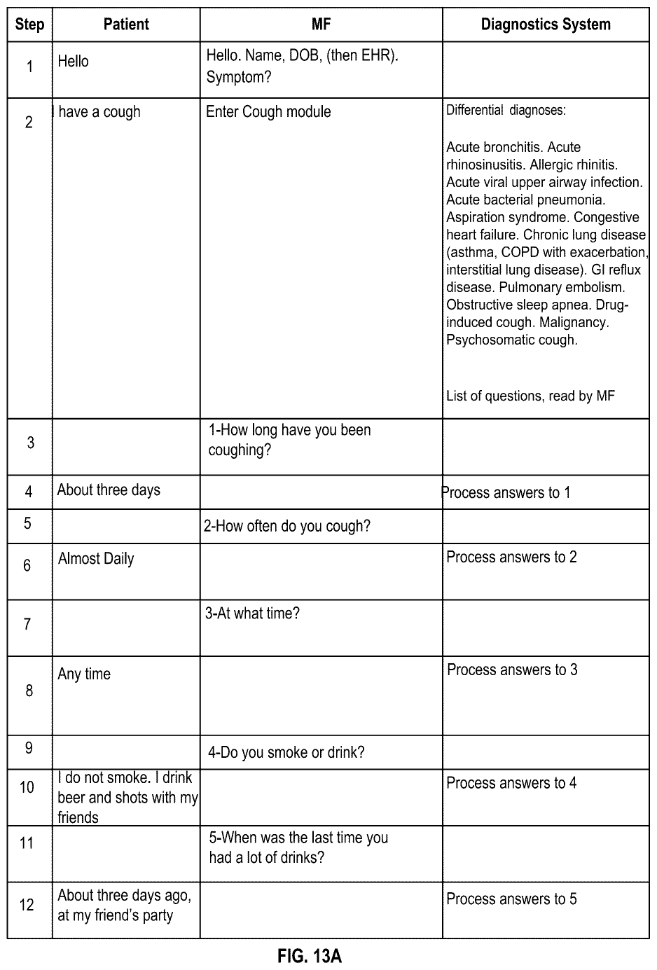

FIGS. 12A-12C are an exemplary illustration of a process for determining and providing a diagnosis of pulmonary embolism using a multi-person interface, in accordance with various examples of the disclosure. In this example, cough symptoms generate multiple differential or potential diagnoses generated by, e.g., the above described neural networks executed by, for example, diagnostics server 202. As the input symptoms are the same as those entered in FIGS. 11A-11C, the output differential diagnoses are also the same or similar. Also generated are a list of questions to be asked by MF 102, for exclusion of various differential diagnoses. The explanation or transmission of these questions by MF 102 to patient 100 is shown in the following several steps, with each question resulting in a patient 100 answer that is entered to diagnostics system 104 by MF 102. The end result of these questions and answers is a final diagnosis of pulmonary embolism. Treatment instructions are then listed as shown, for the MF 102 to relay to patient 100. For example, as pulmonary embolism is a serious and time-critical diagnosis, the MF 102 may be instructed to inform the patient 100 to call 911 and go to the nearest ER immediately.

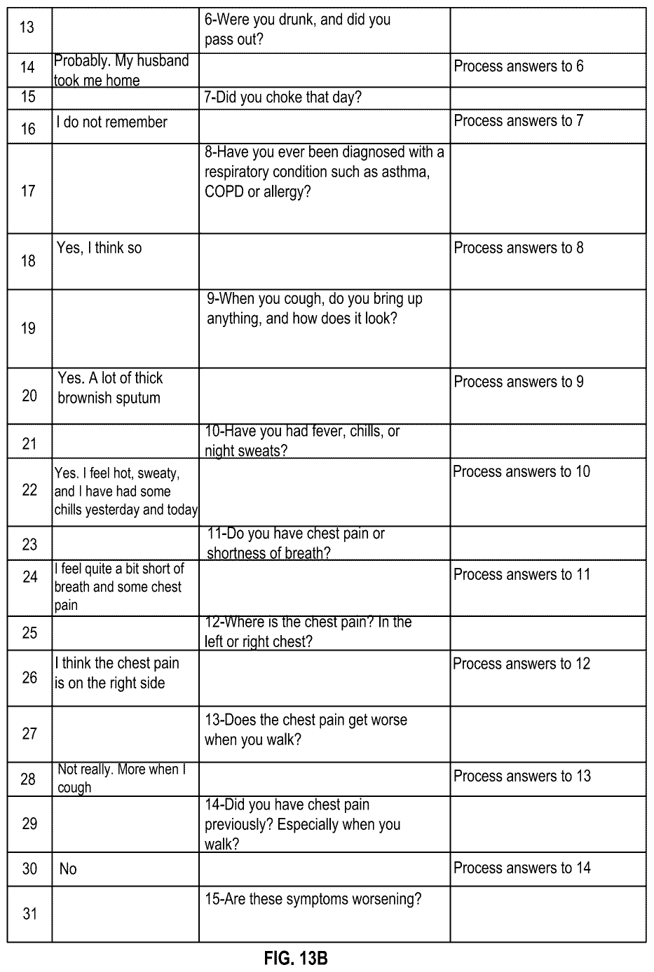

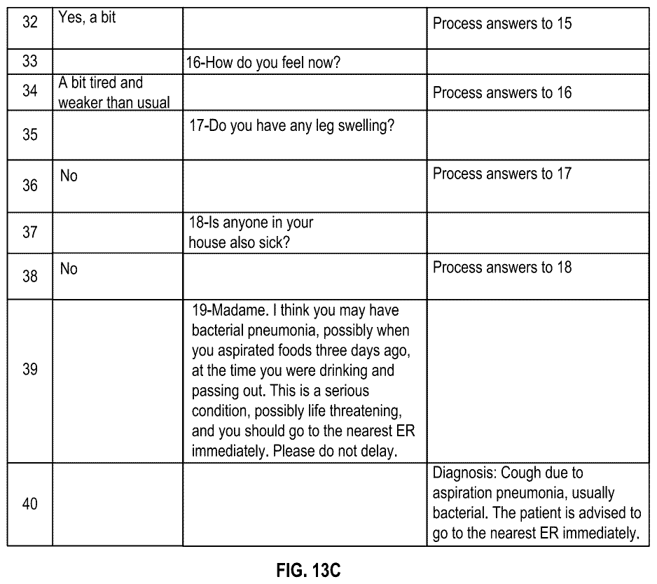

FIGS. 13A-13C are an exemplary illustration of a process for determining and providing a diagnosis of aspiration pneumonia using a multi-person interface, in accordance with various examples of the disclosure. In this example, cough symptoms generate multiple differential or potential diagnoses generated by, e.g., the above described neural networks executed by, for example, diagnostics server 202. As the input symptoms are the same as those entered in FIGS. 11A-11C and FIGS. 12A-12C, the output differential diagnoses are also the same or similar. Also generated are a list of questions to be asked by MF 102, for exclusion of various differential diagnoses. The explanation or transmission of these questions by MF 102 to patient 100 is shown in the following several steps, with each question resulting in a patient 100 answer that is entered to diagnostics system 104 by MF 102. The end result of these questions and answers is a final diagnosis of aspiration pneumonia. Treatment instructions are then listed as shown, for the MF 102 to relay to patient 100. For example, as aspiration pneumonia is a serious and time-sensitive diagnosis, the MF 102 may be instructed to inform the patient 100 to go to the nearest ER immediately.

What is claimed is:

1. A method, comprising:
at an electronic device having one or more processors:
receiving, from a first person, first user input describing symptoms experienced by a second person;
determining a plurality of candidate diagnoses for the described symptoms, the plurality of candidate diagnoses determined according to a neural network trained to receive the described symptoms as inputs and to generate the candidate diagnoses as outputs;
generating questions to exclude ones of the candidate diagnoses;
displaying the generated questions to the first person, for responses by the second person;
receiving, from the first person, second user input describing the responses by the second person;
selecting, according to the second user input, a final diagnosis from among the candidate diagnoses;
transmitting the final diagnosis for confirmation by a third person;
upon confirmation of the final diagnosis by the third person, generating for the second person instructions according to the final diagnosis; and
transmitting the generated instructions for display to the first person, the instructions for execution by the second person.

2. The method of claim 1, wherein the first person is a medical facilitator, the second person is a patient, and the third person is a medical doctor.

3. The method of claim 1, wherein the first user input further describes one or more of a portion of an electronic health record (EHR) of the second person or one or more vital signs of the second person, and the inputs of the neural network further include at least one of the portion of the EHR or the one or more vital signs.

4. The method of claim 1, further comprising administering an appropriate treatment to the second person in accordance with the final diagnosis.

5. The method of claim 4, wherein the administering is performed by one or more of the first person or the third person.

6. The method of claim 1, wherein the neural network comprises one or more machine learning models, the one or more machine learning models including at least one generative model.

7. The method of claim 6, wherein the at least one generative model is trained to generate an image corresponding to the final diagnosis.

8. The method of claim 6, wherein the at least one generative model includes a large language model trained to facilitate a diagnosis from input audio of a patient.

9. The method of claim 1, wherein the neural network comprises one or more machine learning models, the one or more machine learning models including one or more of a classifier model, a regression model, or a large language model (LLM).
